(19) United States
(12) Patent Application Publication
(10) Pub. No.: US 2019/0358264 A1
(43) Pub. Date: Nov. 28, 2019

(54) METHODS OF TREATING DISEASES ASSOCIATED WITH ILC2 CELLS

(71) Applicant: Limm Therapeutics, SA, Paris (FR)

(72) Inventors: José Henrique Veiga Fernandes, Oeiras (PT); Vânia Rita De Faria Cardoso, Guimarães (PT); Julie Michelle Evelyne Chesné, Lisboa (PT)

(73) Assignee: Limm Therapeutics, SA, Paris (FR)

(21) Appl. No.: 16/469,189

(22) PCT Filed: Mar. 29, 2017
(86) PCT No.: PCT/IB2017/000413

§ 371 Date: Jun. 13, 2019

(30) Foreign Application Priority Data
Dec. 13, 2016 (PT) 20161000089304

(51) Int. Cl. A61K 35/17; C12N 5/0783; C07K 16/18; A61K 31/7105; C12N 15/113

(52) U.S. Cl. CPC A61K 35/17; C12N 5/0636; C07K 16/18; C07K 2317/76; C12N 15/113; C12N 2501/80; A61K 31/7105

(57) ABSTRACT

Provided herein are compositions including compounds and/or cells for treating a disease associated with Group 2 innate lymphoid cells (ILC2s), and methods of treatment.

### BACKGROUND

Group 2 innate lymphoid cells (ILC2s) are abundant at mucosal barriers and act as key initiators of type 2 inflammation and tissue repair^{1,2}. ILC2s are activated by cell-extrinsic cytokines, including IL-25, IL-33 and thymic stromal lymphopoietin^{1,2}. Previous reports indicated that discrete lymphocyte subsets and haematopoietic progenitors are controlled by dietary signals and neuroregulators^{2,3,5-9}, suggesting that ILC2s may exert their function in the context of neuro-immune cell units.

### SUMMARY

As shown herein, the neuropeptide Neuromedin U has been determined to be a uniquely potent regulator of type 2 innate immunity in the context of a novel neuron-ILC2 unit. More specifically, it was determined that ILC2s express the Neuromedin U receptor 1 (Nmur1) while Neuromedin U is expressed by enteric neurons. Activation of ILC2s with Neuromedin U resulted in prompt and strong production of the type 2 cytokines interleukin 5 (IL-5), IL-13 and Amphiregulin in a NMUR1-dependent manner. Neuromedin U controlled ILC2 downstream of ERK activation and calcium-influx-dependent activation of Calcineurin cytokines and NFAT. Moreover, Neuromedin U treatment in vivo resulted in immediate type 2 responses. Accordingly, ablation of Nmur1 led to impaired type 2 responses and poor worm infection control. Strikingly, mucosal neurons were found adjacent to ILC2s and directly sensed worm products to control Neuromedin U expression and innate type 2 cytokines. This work reveals novel neuro-immune interactions at the core of mucosal homeostasis indicating that neuron-ILC2 cell units are poised to confer immediate protection via coordinated neuro-immune sensory responses.

According to one aspect, methods for increasing activity or proliferation of Group 2 innate lymphoid cells (ILC2s) are provided. The methods include contacting ILC2s with an agonist of neuromedin U receptor 1 (NMUR1) in an amount effective to increase activity of the ILC2s. In some embodiments, the agonist of NMUR1 is neuromedin U (NMU) or an analog thereof, or an antibody that specifically binds and activates NMUR1 or an antigen-binding fragment thereof. In some embodiments, the NMU or analog thereof is NMU25, NMU precursor protein, NMU23, or NMU8.

In some embodiments, the contacting is in vitro. In some embodiments, the ILC2s are contacted in an ILC2 expansion protocol.

In other embodiments, the contacting is in vivo. In some embodiments, the agonist of neuromedin U receptor 1 (NMUR1) is administered to a subject. In some embodiments, the subject is a human. In some embodiments, the subject is not otherwise in need of treatment with the agonist of NMUR1.

According to another aspect, methods for treating a disease associated with Group 2 innate lymphoid cells (ILC2s) are provided. In some embodiments, the methods include administering to a subject in need of such treatment an agonist of neuromedin U receptor 1 (NMUR1) in an amount effective to treat the disease. In some embodiments, the agonist of NMUR1 is neuromedin U (NMU) or an analog thereof, or an antibody that specifically binds and activates NMUR1 or an antigen-binding fragment thereof. In some embodiments, the NMU or analog thereof is NMU25, NMU precursor protein, NMU23, or NMU8. In some embodiments, the subject is a human.

In some embodiments, the disease is infection, tissue repair, wound healing, obesity, treatable by increasing induction of type 2 immune responses, treatable by metabolic regulation, treatable by increasing eosinophils, or treatable by increasing mast cells. In some embodiments, the subject is not otherwise in need of treatment with the agonist of NMUR1.

In some embodiments, the agonist of NMUR1 is administered intravenously, orally, nasally, rectally or through skin absorption.

According to another aspect, agonists of neuromedin U receptor 1 (NMUR1) are provided for use in treating a disease associated with Group 2 innate lymphoid cells (ILC2s) including administering to a subject in need of such treatment the agonist of NMUR1 in an amount effective to treat the disease. In some embodiments, the agonist of NMUR1 is neuromedin U (NMU) or an analog thereof, or an antibody that specifically binds and activates NMUR1 or an antigen-binding fragment thereof. In some embodiments, the NMU or analog thereof is NMU25, NMU precursor protein, NMU23, or NMU8. In some embodiments, the subject is a human.

In some embodiments, the disease is infection, tissue repair, wound healing, obesity, treatable by increasing induction of type 2 immune responses, treatable by metabolic regulation, treatable by increasing eosinophils, or treatable by increasing mast cells. In some embodiments, the subject is not otherwise in need of treatment with the agonist of NMUR1.

In some embodiments, the agonist of NMUR1 is administered intravenously, orally, nasally, rectally or through skin absorption.

According to another aspect, methods for treating a disease associated with Group 2 innate lymphoid cells (ILC2s) are provided. The methods include administering to a subject in need of such treatment a composition comprising activated ILC2s in an amount effective to treat the disease. In some embodiments, the composition further comprises an agonist of neuromedin U receptor 1 (NMUR1). In some embodiments, the agonist of NMUR1 is neuromedin U (NMU) or an analog thereof, or an antibody that specifically binds and activates NMUR1 or an antigen-binding fragment thereof. In some embodiments, the NMU or analog thereof is NMU25, NMU precursor protein, NMU23, or NMU8. In some embodiments, the subject is a human.

In some embodiments, the disease is infection, tissue repair, wound healing, obesity, treatable by increasing induction of type 2 immune responses, treatable by metabolic regulation, treatable by increasing eosinophils, or treatable by increasing mast cells. In some embodiments, the subject is not otherwise in need of treatment with the activated ILC2s or the agonist of NMUR1.

In some embodiments, the activated ILC2s or the agonist of NMUR1 is administered intravenously, orally, nasally, rectally or through skin absorption.

According to another aspect, compositions are provided that include activated Group 2 innate lymphoid cells (ILC2s) for use in treating a disease associated with ILC2s including administering to a subject in need of such treatment the composition comprising activated ILC2s in an amount effective to treat the disease. In some embodiments, the composition further comprises an agonist of neuromedin U receptor 1 (NMUR1). In some embodiments, the agonist of NMUR1 is neuromedin U (NMU) or an analog thereof, or an antibody that specifically binds and activates NMUR1 or an antigen-binding fragment thereof. In some embodiments, the NMU or analog thereof is NMU25, NMU precursor protein, NMU23, or NMU8. In some embodiments, the subject is a human.

In some embodiments, the disease is infection, tissue repair, wound healing, obesity, treatable by increasing induction of type 2 immune responses, treatable by metabolic regulation, treatable by increasing eosinophils, or treatable by increasing mast cells. In some embodiments, the subject is not otherwise in need of treatment with the activated ILC2s or the agonist of NMUR1.

In some embodiments, the activated ILC2s or the activated ILC2s and the agonist of NMUR1 is administered intravenously, orally, nasally, rectally or through skin absorption.

According to another aspect, methods for decreasing activity or proliferation of Group 2 innate lymphoid cells (ILC2s) are provided. The methods include contacting ILC2s with an antagonist of neuromedin U receptor 1 (NMUR1) or neuromedin U (NMU) in an amount effective to decrease activity of the ILC2s. In some embodiments, the antagonist of NMUR1 or NMU is an antibody that specifically binds and inhibits NMUR1 or NMU, respectively, or an antigen-binding fragment thereof. In some embodiments, the antagonist of NMUR1 or NMU is an inhibitory nucleic acid molecule that reduces that reduces expression, transcription or translation of NMUR1 or NMU. In some embodiments, the inhibitory nucleic acid is a sRNA, shRNA, or antisense nucleic acid molecule.

In some embodiments, the contacting is in vitro.

In other embodiments, the contacting is in vivo. In some embodiments, the antagonist of NMUR1 or NMU is administered to a subject. In some embodiments, the subject is a human. In some embodiments, the subject is not otherwise in need of treatment with the antagonist of NMUR or NMU1.

According to another aspect, methods for treating a disease associated with Group 2 innate lymphoid cells (ILC2s) are provided. The methods include administering to a subject in need of such treatment an antagonist of neuromedin U receptor 1 (NMUR1) or neuromedin U (NMU) in an amount effective to treat the disease. In some embodiments, the antagonist of NMUR1 or NMU is an antibody that specifically binds and inhibits NMUR1 or NMU, respectively, or an antigen-binding fragment thereof. In some embodiments, the antagonist of NMUR1 or NMU is an inhibitory nucleic acid molecule that reduces that reduces expression, transcription or translation of NMUR1 or NMU. In some embodiments, the inhibitory nucleic acid is a sRNA, shRNA, or antisense nucleic acid molecule. In some embodiments, the subject is a human.

In some embodiments, the disease is allergy, allergic asthma, food allergy, eosinophilic esophagitis, atopic dermatitis, fibrosis, allergic rhinitis, allergic rhinosinusitis, chronic obstructive pulmonary disease (COPD), cystic fibrosis, treatable by reducing type 2 immune responses, treatable by reducing eosinophils, or treatable by reducing mast cells. In some embodiments, the subject is not otherwise in need of treatment with the agonist of NMUR1 or NMU.

In some embodiments, the antagonist of NMUR1 is administered intravenously, orally, nasally, rectally or through skin absorption.

According to another aspect, antagonists of neuromedin U receptor 1 (NMUR1) or neuromedin U (NMU) are provided for use in treating a disease associated with Group 2 innate lymphoid cells (ILC2s) comprising administering to a subject in need of such treatment the antagonist of NMUR1 or NMU in an amount effective to treat the disease. In some embodiments, the antagonist of NMUR1 or NMU is an antibody that specifically binds and inhibits NMUR1 or NMU, respectively, or an antigen-binding fragment thereof. In some embodiments, the antagonist of NMUR1 or NMU is an inhibitory nucleic acid molecule that reduces that reduces expression, transcription or translation of NMUR1 or NMU. In some embodiments, the inhibitory nucleic acid is a sRNA, shRNA, or antisense nucleic acid molecule. In some embodiments, the subject is a human.

In some embodiments, the disease is allergy, allergic asthma, food allergy, eosinophilic esophagitis, atopic dermatitis, fibrosis, allergic rhinitis, allergic rhinosinusitis, chronic obstructive pulmonary disease (COPD), cystic fibrosis, treatable by reducing type 2 immune responses, treatable by reducing eosinophils, or treatable by reducing mast cells. In some embodiments, the subject is not otherwise in need of treatment with the agonist of NMUR1 or NMU.

In some embodiments, the antagonist of NMUR1 or NMU is administered intravenously, orally, nasally, rectally or through skin absorption.

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1a-1e. ILC2s express neuromedin U receptor 1 and closely locate with Neuromedin U-expressing neurons. FIG. 1a, Heat map for 40 neuronal-related mRNA transcripts in CD4 T cells, ILC1s, ILC2s, NCR⁻ (CD4⁺ and CD4⁻) and NCR⁺ ILC3s subsets¹⁰. FIG. 1b, Comparison of ILC2 gene expression with ILC1, ILC3 NCR⁺ and CD4 T cells¹⁰, by volcano plots. Nmur1 is highlighted in red. FIG. 1c, Nmur1 quantitative RT-PCR analysis in intestinal lamina propria cells unless stated otherwise. Common lymphoid progenitor (CLP); Common helper innate lymphoid progenitor (CHILP); Bone marrow ILC2 progenitor (ILC2P); Eosinophils (Eo); Mast cells (Mast); Macrophages (Mø); Neutrophils (Neu); Dendritic cells (DC); T cells (T); B cells (B); Lamina propria glial cells (G) and neurons (N); Epithelial cells (Ep). n=6. FIG. 1d, Nmu quantitative RT-PCR analysis in intestinal populations. n=6. FIG. 1e, Confocal analysis of intestinal lamina propria. Green: neurons (Ret^{GFP}); Red: KLRG1; Cyan: CD3. Cyan arrows: T cells (CD3⁺). Red arrows: ILC2s.

FIGS. 2a-2j. Neuromedin U is a uniquely potent regulator of innate type 2 cytokines, via NMUR1 activation. FIGS. 2a-2f, ILC2-intrinsic activation with NmU23. FIG. 2a, Type 2 cytokine gene expression in intestinal ILC2s. n=6. FIG. 2b, Type 2 cytokine gene expression in lung ILC2s. n=6. FIG. 2c, Ki67 expression in intestinal ILC2s. FIG. 2d, IL-5 and IL-13 expression in Nmur1 competent and deficient ILC2s. FIG. 2e, Innate inflammatory type 2 cytokines at the protein level. n=6. FIG. 2f, Innate tissue-repair cytokine AREG. n=6. FIGS. 2g,2h, in vivo administration of NmU23. FIG. 2g, ILC2-derived type 2 cytokines. n=6. FIG. 2h, T cell-derived type 2 cytokines. n=6. FIGS. 2i,2j, in vivo ablation of Nmur1. FIG. 2i, Intestinal ILC2s from Nmur1^{-/-} and their Nmur1^{+/+} WT littermate controls. WT n=6; Nmur1^{-/-} n=9. FIG. 2j, ILC2-derived type 2 cytokines in bone marrow chimeras of Nmur1^{-/-} and in their Nmur1^{+/+} WT littermate control origin. WT n=6; Nmur1^{-/-} n=3. Error bars show s.e.m. *P<0.05; **P<0.01; ***P<0.001; ****P<0.0001; ns not significant.

FIGS. 3a-3e. Neuromedin U regulates ILC2-derived cytokines via ERK1/2 and a Ca²⁺/Calcineurin/NFAT cascade. FIGS. 3a-e, Intestinal ILC2 activation by Neuromedin U. FIG. 3a, Percentage of pERK cells n=4. Mean fluorescence intensity (MFI) of pERK expression. n=4. FIG. 3b, Il5, Il13 and Csf2 expression in ILC2s cultured with medium (control) (n=3), NmU23 (n=3) or NmU23 and ERK inhibitor PD98059 (n=3). FIG. 3c, Left and centre: Ca²⁺ influx, represented by Fluo-4 AM intensity. NmU23 was added 60 seconds after ILC2 baseline acquisition (arrow). Right: Mean intensity of Ca²⁺ influx. n=3. FIG. 3d, Il5, Il13 and Csf2 expression in ILC2s cultured with medium (control) (n=6), NmU23 (n=6) or NmU23 and Calcineurin inhibitor FK506 (n=6). FIG. 3e, Il5, Il13 and Csf2 expression in ILC2s cultured with medium (control) (n=3), NmU23 (n=3) or NmU23 and NFAT inhibitor 11R-VIVIT (VIVIT) (n=3). Error bars show s.e.m. *P<0.05; **P<0.01; ***P<0.001; ****P<0.0001; ns not significant.

FIGS. 4a-4h. The neuroregulatory axis NmU-NMUR1 confers protection against worm infection. Mice were infected with N. brasiliensis larvae and lungs analysed at 48 hours. FIG. 4a, Nmu expression in total lung from infected mice compared to non-infected controls. n=3. FIG. 4b, Pulmonary inflammatory cell infiltrates 48 hours after infection. NmU23 treated and control sections are displayed. Hematoxylin and eosin. FIG. 4c, Myeloperoxidase- (granulocytes) and Luna-stained (eosinophils) lung sections. FIG. 4d, Granulocyte and eosinophil cell counts (cells/mm²). Control n=8; NmU23 n=8. FIG. 4e, Nmur1^{-/-} and their WT littermate controls were infected with N. brasiliensis. Hematoxylin and eosin. FIG. 4f, Myeloperoxidase- (granulocytes) and Luna-stained (eosinophils) lung sections. FIG. 4g, Granulocyte and eosinophilic cell counts (cells/mm²). WT n=8; Nmur1^{-/-} n=8. FIG. 4h, N. brasiliensis infection burden at 48 hours in the lung. WT n=3; Nmur1^{-/-} n=3. Scale bars: 50 μm. Error bars show s.e.m. *P<0.05; **P<0.01; ***P<0.001; ****P<0.0001; ns not significant.

FIGS. 5a-5c. Genome-wide ILC2 transcriptional profiling and neuron-ILC2 interactions. FIG. 5a, Weighted Unifrac PCoA analysis of ILC2s, CD4 T cells, ILC1s and ILC3s. FIG. 5b, Levels of Nmur1 expression in ILC2s, CD4 T cell, ILC1 and ILC3 populations. FIG. 5c, Separate channels of confocal analysis in FIG. 1e right. Green: neurons (Ret^{GFP}); Red: KLRG1; Cyan: CD3.

FIGS. 6a-6f. Neuromedin U is potent regulator of lung innate type 2 cytokines, via NMUR1 activation. FIGS. 6a,6b, ILC2-intrinsic activation with NmU23. FIG. 6a, IL-5 and IL-13 expression in lung ILC2s. FIG. 6b, Innate type 2 cytokines at the protein level. n=3.

FIGS. 6c,6d, in vivo administration of NmU23. FIG. 6c, ILC2-derived type 2 cytokines in the lung. n=3. FIG. 6d, T cell-derived type 2 cytokines in the lung. n=3. FIGS. 6e,6f, in vivo ablation of Nmur1. FIG. 6e, Lung ILC2s in Nmur1^{-/-} and in their Nmur1^{+/+} WT littermate controls. WT n=6; Nmur1^{-/-} n=9. FIG. 6f, Intestinal T cell-derived type 2 cytokines in Nmur1^{-/-} and in their Nmur1^{+/+} WT littermate controls. WT n=6; Nmur1^{-/-} n=6. Error bars show s.e.m. *P<0.05; **P<0.01; ***P<0.001; ****P<0.0001; ns not significant.

FIGS. 7a-7c. Nmur1 is dispensable for ILC2 development. FIGS. 7a,7c, Competitive bone marrow chimeras. FIG. 7a, 10⁶ cells of each genotype (CD45.2) were injected intravenously in direct competition with a third-party WT competitor (CD45.1/CD45.2), in a 1:1 ratio, into non-lethally irradiated (150 Rad) NSG mice (CD45.1). FIG. 7b, Percentage and number of donor ILC2s in the intestine. WT n=12; Nmur1^{-/-} n=12. FIG. 7c, Percentage and number of donor ILC2s in the lung. WT n=12; Nmur1^{-/-} n=12. Error bars show s.e.m. *P<0.05; **P<0.01; ***P<0.001; ****P<0.0001; ns not significant.

FIG. 8. A novel neuron-ILC2 unit orchestrated by neuromedin U. Neuron-derived Neuromedin U directly activates ILC2s in a NMUR1 dependent manner, resulting in a potent production of inflammatory and tissue repair type 2 cytokines that confer protection to worm infection. Neuromedin U activates NMUR1 with induces type 2 cytokine expression downstream of ERK phosphorylation and activation of a Ca²⁺/Calcineurin/NFAT cascade. This model suggests that neuron-ILC2 cell units are poised to uniquely ensure potent and immediate type 2 responses in a neuromedin U-dependent manner.

FIGS. 9a-9i: FIG. 9a, Nmur1 quantitative RT-PCR analysis in the lungs at day 6 post Nippostrongylus brasiliensis (NB)—infection in lung. Eosinophils (Eo); Mast cells (Mast); Macrophages (Mø); Neutrophils (Neu); naive T cells (T); Innate lymphoid cells type 2 (ILC2). FIG. 9b, NMUR1 expression in human adaptive (CD4 T cells) and innate type 2 lymphocytes ILC2 from blood. FIG. 9c, Type 2 cytokine gene expression in human ILC2 and Th2 after in vitro stimulation with the peptide NmU25. FIG. 9d, Nmur1 expression in lung ILC2 before and after infection (at day 6). FIG. 9e, Nmur1 expression in steady state in Common lymphoid progenitor (CLP); Common helper innate lymphoid progenitor (CHILP), Bone marrow ILC2 progenitor (ILC2P) and Eo, Mast, Mø, Neu, Dendritic cells (DC); naive T cells (T); T-helper 2 cells (Th2); memory T cells, B cells (B), Lamina Propria glial cells (G) and neurons (N). n=3-6. FIG. 9f, Type 2 cytokine gene expression in intestinal ILC2 and Th2 after in vitro stimulation with the peptide NmU23 (100 ng/mL). n=3-6. FIG. 9g, Confocal analysis of intestinal lamina propria. Green: neurons (Ret^{GFP}); Cyan: KLRG1; red: CD3. Cyan: KLRG1. FIG. 9h, Neurosphere-derived neurons. Red: TUJ1. Blue: DAPI. FIG. 9i, Activation of neurosphere-derived neurons with alarmins, TLR-ligands and N. brasiliensis excretory/secretory proteins (NES). *P<0.05; **P<0.01; ***P<0.001; ****P<0.0001; ns not significant.

FIGS. 10a-10d: FIG. 10a, Ki67 expression in intestinal ILC2s after an overnight in vitro stimulation with NmU23 alone (100 ng/mL, Phoenix Pharmaceutical) or NmU23 together with the survival cytokines Interleukin (IL)-2 and/or IL-7 (10 ng/mL). FIG. 10b, Ki67 expression in intestine ILC2 after in vivo administration of NmU23 (4 g/day during 2 days). n=5. FIG. 10c, ILC2-derived type 2 cytokines (IL-5, IL-13 and Amphiregulin (Areg)) in sorted intestine ILC2 after an overnight stimulation with NmU23, mouse recombinant IL-25 or IL-33 (R&D) (10, 50 and 100 ng/mL). Negative control: unstimulated ILC2, Positive control: ILC2 activated with phorbol 12-myristate 13-acetate (PMA, 50 ng/ml) plus ionomycin (500 ng/ml). n=3. FIG. 10d, Dot plots representative of the cytokine production with increasing dose of NmU23, rIL-25 and rIL-33. *P<0.05; **P<0.01; ***P<0.001; ****P<0.0001; ns not significant.

FIGS. 11a-11b: ILC2 were FBS deprived for 2 hours prior to treatment with either (FIG. 11a) 11R-VIVIT (inhibits NFAT activation) (10 μM) or (FIG. 11b) cyclosporin A (CsA, 100M). Expression of type 2 cytokines were measured by quantitative RT-PCR (FIGS. 11a,11b). n=3-6. FIG. 11c, Deprivated ILC2 from Lamina Propria were stimulated 90′ with NmU23 (100 ng/mL), fixed, permeabilized and stained with anti-NFAT2 monoclonal antibody (abcam). Cells were analyzed by confocal microscopy. *P<0.05; **P<0.01; ***P<0.001; ****P<0.0001; ns not significant.

FIGS. 12a-12f: (FIGS. 12a-12c) Mice were infected with N. brasiliensis larvae and treated with NmU23 peptide (8 g/day) or PBS (control). Lungs were analysed at day 2 post-infection. FIG. 12a, ILC2 response in lungs from NmU23 treated mice (n=5) compared to control (n=5). FIG. 12b, Burden of infection in lungs of infected mice treated with PBS (n=5) or NmU23 (n=5). FIG. 12c, Pulmonary hemorrhage in lung of infected mice treated with NmU23 compared to control. (FIGS. 12d-12f) Mice were infected with N. brasiliensis larvae and treated with NmU23 peptide (8 g/day) or PBS (control). Lungs and small intestine were analysed at day 6 post-infection. FIG. 12d, Neutrophils and eosinophils infiltrate in broncho-alveolar lavage (BAL) in infected mice treated with NmU23 versus PBS. Control n=5; NmU23 n=5. FIG. 12e, Mastocytes and Macrophages infiltrate in broncho-alveolar lavage (BAL) in infected mice treated with NmU23 versus PBS. Control n=5; NmU23 n=5. FIG. 12f, Burden of infection in small intestine of infected mice treated with PBS (n=5) or NmU23 (n=5). Error bars show s.e.m. *P<0.05; **P<0.01; ***P<0.001; ****P<0.0001; ns not significant.

FIGS. 13a-13c: Nmur1^{-/-} and their WT littermate controls were infected with N. brasiliensis and analyzed at day 6 post-infection. FIG. 13a, ILC2 response in lungs of infected Nmur1^{-/-} and their WT littermate controls D6 post-infection. WT n=6; Nmur1^{-/-} n=8. FIG. 13b, Neutrophils (Neu) and eosinophils (Eos) infiltrate in broncho-alveolar lavage (BAL) in infected Nmur1^{-/-} and their WT littermate controls. WT n=6; Nmur1^{-/-} n=7. FIG. 13c, Mastocytes and Macrophages infiltrate in broncho-alveolar lavage (BAL) in infected Nmur1^{-/-} and their WT littermate controls. WT n=6; Nmur1^{-/-} n=7. Error bars show s.e.m. *P<0.05; **P<0.01; ***P<0.001; ****P<0.0001; ns not significant.

FIGS. 14a-14c: Competitive bone marrow chimeras treated with NmU23. FIG. 14a, 10⁶ cells of each genotype (CD45.2) were injected intravenously in direct competition with a third-party WT competitor (CD45.1/CD45.2), in a 1:1 ratio, into non-lethally irradiated (150 Rad) NSG mice (CD45.1). The mice received one injection of PBS or NmU23 (20 μg). FIG. 14b, Percentage and number of donor ILC2s in the lungs. WT n=5; Nmur1^{-/-} n=5. FIG. 14c, Percentage and number of donor T cells in the lungs. WT n=5; Nmur1^{-/-} n=5. Error bars show s.e.m. *P<0.05; **P<0.01; ***P<0.001; ****P<0.0001; ns not significant.

### DETAILED DESCRIPTION

Group 2 innate lymphoid cells (ILC2s) are major regulators of inflammation, tissue repair and metabolic homeostasis^{1,2}. ILC2 activation has been shown by host-derived cytokines and alarmins^{1,2}, but, how ILC2s respond to neuronal-derived signals remains unclear.

As described herein, it was determined that ILC2s express the Neuromedin U receptor 1 (Nmur1) and that the neuropeptide Neuromedin U is a potent activator of ILC2s. Neuromedin U resulted in prompt and strong production of the type 2 cytokines interleukin 5 (IL-5), IL-13 and Amphiregulin in a NMUR1-dependent manner. Neuromedin U controlled ILC2 downstream of ERK activation and calcium-influx-dependent activation of Calcineurin cytokines and NFAT. When used in vivo, Neuromedin U treatment resulted in immediate type 2 responses. It also was shown that ablation of Nmur1 led to impaired type 2 responses and poor worm infection control.

### Increasing Activity of ILC2s

The methods disclosed herein include methods for increasing activity or proliferation of Group 2 innate lymphoid cells (ILC2s) by contacting ILC2s with an agonist of neuromedin U receptor 1 (NMUR1) in an amount effective to increase activity of the ILC2s.

The methods disclosed herein also include methods for treating a disease associated with Group 2 innate lymphoid cells (ILC2s) by administering to a subject in need of such treatment an agonist of neuromedin U receptor 1 (NMUR1) in an amount effective to treat the disease.

Other methods for treating disease include administering to a subject in need of such treatment a composition comprising activated ILC2s in an amount effective to treat the disease. In some of these methods, the composition comprising activated ILC2s also includes an agonist of neuromedin U receptor 1 (NMUR1). Alternatively, an agonist of NMUR1 can be administered separately from the composition comprising activated ILC2s.

Also provided herein are agonists of NMUR1 for use in treating a disease associated with ILC2s, and compositions comprising ILC2s (and optionally an agonist of NMUR1) for use in treating a disease associated with ILC2s.

As used herein, neuromedin U receptor 1 (NMUR1) is a 7 transmembrane receptor of the rhodopsin family, and is also known as FM3, FM-3, GPC-R, G-protein coupled receptor 66 (GPR66), and NMU1R. As described elsewhere herein, an agonist of NMUR1 includes a neuromedin U (NMU) or an analog thereof, an antibody that specifically binds and activates NMUR1 or an antigen-binding fragment thereof, or a small molecule ligand of NMUR1.

Contacting ILC2s with an agonist of NMUR1 can be performed in vitro, such as in an ILC2 expansion protocol performed to produce ILC2s, or can be performed in vivo. In some embodiments of methods in which the contacting of ILC2s with an agonist of NMUR1 is performed in vivo, the agonist of NMUR1 is administered to a subject, such as a human. In some of these methods, the subject is not otherwise in need of treatment with the agonist of NMUR1.

In the disclosed methods, the subject can be a human. In some of these methods, the subject is not otherwise in need of treatment with the agonist of NMUR1 and/or treatment with the activated ILC2s.

Diseases treatable by the disclosed methods include infection, tissue repair, wound healing, obesity, diseases treatable by increasing induction of type 2 immune responses, diseases treatable by metabolic regulation, diseases treatable by increasing eosinophils, and diseases treatable by increasing mast cells.

The agonist of NMUR1 and/or the activated ILC2s can be administered by any suitable route of administration or delivery method. Suitable routes of administration include intravenous, oral, nasal, rectal or through skin absorption.

The agonist of NMUR1 and/or the activated ILC2s can be administered at any suitable interval, including daily, twice daily, three times per day, four times per day, every other day, weekly, every two weeks, every four weeks, continuously (e.g., by infusion, patch, or pump), and so on.

### Decreasing Activity of ILC2s

Additional methods disclosed herein include methods for decreasing activity or proliferation of Group 2 innate lymphoid cells (ILC2s) by contacting ILC2s with an antagonist of neuromedin U receptor 1 (NMUR1) or an antagonist of NMU (or both) in an amount effective to decrease activity of the ILC2s.

The methods disclosed herein also include methods for treating a disease associated with Group 2 innate lymphoid cells (ILC2s) by administering to a subject in need of such treatment an antagonist of neuromedin U receptor 1 (NMUR1) in an amount effective to treat the disease.

Also provided herein are antagonists of NMUR1 for use in treating a disease associated with ILC2s.

As described elsewhere herein, an antagonist of NMUR1 includes an inhibitory nucleic acid molecule that reduces that reduces expression, transcription or translation of NMUR1, such as a sRNA, shRNA, or antisense nucleic acid molecule; an antibody that specifically binds and inhibits NMUR1 or an antigen-binding fragment thereof, or a small molecule antagonist of NMUR1.

Contacting ILC2s with an antagonist of NMUR1 can be performed in vitro, or can be performed in vivo. In some embodiments of methods in which the contacting of ILC2s with an antagonist of NMUR1 is performed in vivo, the antagonist of NMUR1 is administered to a subject, such as a human. In some of these methods, the subject is not otherwise in need of treatment with the antagonist of NMUR1.

In the disclosed methods, the subject can be a human. In some of these methods, the subject is not otherwise in need of treatment with the antagonist of NMUR1.

In the methods disclosed herein for treating disease by administering an antagonist of NMUR1, the disease can be allergy, allergic asthma, food allergy, eosinophilic esophagitis, atopic dermatitis, fibrosis, allergic rhinitis, allergic rhinosinusitis, chronic obstructive pulmonary disease (COPD), cystic fibrosis, diseases treatable by reducing type 2 immune responses, diseases treatable by reducing eosinophils, or diseases treatable by reducing mast cells.

The antagonist of NMUR1 can be administered by any suitable route of administration or delivery method. Suitable routes of administration include intravenous, oral, nasal, rectal or through skin absorption.

The antagonist of NMUR1 can be administered at any suitable interval, including daily, twice daily, three times per day, four times per day, every other day, weekly, every two weeks, every four weeks, continuously (e.g., by infusion, patch, or pump), and so on.

### Agonists of Neuromedin U Receptor 1 (NMUR1)

Agonists of NMUR1 include peptide agonists (including modified peptides and conjugates), activating antibody molecules, and small molecules. Peptide agonists include neuromedin U (also known as and referred to herein as NMU or NmU) or analogs thereof. The NMUR1 agonists may be entirely specific for NMUR1, may agonize NMUR1 preferentially (as compared to neuromedin U receptor 2, NMUR2), or may agonize both NMUR1 and NMUR2. Such agonists may be useful even if NMUR1 is agonized less than NMUR2, but it is preferred that the agonists used in the methods described herein agonize NMUR1 to a greater extent than NMUR2. As used herein agonizing NMUR1 preferentially (as compared to neuromedin U receptor 2, NMUR2) means that the agonist agonizes NMUR1 at least 10%, 25%, 50%, 100%, 200%, 300%, 400%, 500%, 600%, 700%, 800%, 900%, 1000%, or more than NMUR2.

Neuromedin U (also referred to herein as NMU) is a neuropeptide conserved in many species, which was isolated as a peptide consisting of 25 amino acid residues (NMU-25) or as a peptide consisting of 8 amino acid residues (NMU-8), from pig small intestine. NMU-8 consists of the C-terminal 8 residues of porcine NMU25. NMU-25 also is present in humans, and is preferred for use in humans. The C-terminal 8 amino acid residues of human NMU-25 (also referred to as NMU-8) are the same as that of the C-terminal 8 amino acid residues of porcine NMU-8. The 8 amino acids at the C terminus of NMU-25 are the most highly conserved and this peptide has been shown to have similar activity as NMU-25. Rat NMU consists of 23 amino acid residues, and is known as NMU-23. The amino acid sequence of the C-terminal 8 residues of rat NMU-23 differs from that of the C-terminal 8 residues of porcine NMU-8 by one amino acid residue. NMU precursor protein (and its cleaved peptides) also can be used in the methods described herein. NMU precursor protein is a 174 amino acid long protein.

Amino acid sequences of the NMU precursor protein and NMU are provided as follows:

NMU precursor protein
(P48645|NMU_HUMAN Neuromedin-U OS = Homo sapiens
GN = NMU PE = 1 SV = 1)
(SEQ ID NO: 1)
MLRTESCRPRSPAGQVAAASPLLLLLLLLAWCAGACRGAPILPQGLQPEQ
QLQLWNEIDDTCSSFLSIDSQPQASNALEELCFMIMGMLPKPQEQDEKDN
TKRFLFHYSKTQKLGKSNVVSSVVHPLLQLVPHLHERRMKRFRVDEEFQS
PFASQSRGYFLFRPRNGRRSAGFI
NMU25
(SEQ ID NO: 2)
FRVDEEFQSPFASQSRGYFLFRPRN
NMU23
(SEQ ID NO: 3)
FKAEYQSPSVGQSKGYFLFRPRN
NMU8
(SEQ ID NO: 4)
YFLFRPRN

Agonists of NMUR1 include NMU analogs, derivatives, and conjugates, such as NMU analogs having variations in amino acid sequence relative to natural NMU sequences but which retain function of binding to and activating NMUR1. Other examples of analogs, derivatives, and conjugates of NMU include: the modified peptides of Takayama et al. (ACS Med Chem Lett. 2015 Mar. 12; 6(3): 302-307); the NMU-8 analogs of Inooka et al. (Bioorg Med Chem. 2017 Feb. 21. pii: S0968-0896(17)30108-6); the PEGylated derivatives of NMU of Ingallinella et al. (Bioorg Med Chem. 2012 Aug. 1; 20(15):4751-9); the human serum albumin (HSA)-NMU conjugate of Neuner et al. (J Pept Sci. 2014 January; 20(1):7-19); the truncated/lipid-conjugated NMU analogs of Micewicz (Eur J Med Chem. 2015 Aug. 28; 101:616-26); and the lipidated NMU analogs of Dalbege et al. (J Pept Sci. 2015 February; 21(2):85-94).

As described in US 2011/0294735 and WO 2007/109135 (each incorporated herein by reference for the specific recitation of the following compounds), additional NMUR1 agonists comprise the general formula (I)

Z¹-peptide-Z² (I)

wherein the peptide has the amino acid sequence X¹-X²-X³-X⁴-X⁵-X⁶-X7-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵, wherein amino acids 1 to 17 can be any amino acid or absent, wherein amino acid X¹⁸ is absent, Y, W, F, a des-amino acid or an acyl group; amino acid X¹⁹ is A, W, Y, F or an aliphatic amino acid; amino acid X²⁰ is absent, L, G, sarcosine (Sar), D-Leu, NMe-Leu, D-Ala or A; amino acid X²¹ is F, NMe-Phe, an aliphatic amino acid, an aromatic amino acid, A or W; X²² is R, K, A or L; amino acid X²³ is P, Sar, A or L; amino acid X²⁴ is R, Harg or K; and amino acid X²⁵ is N, any D- or L-amino acid, Nle or D-Nle, A; and Z¹ is an optionally present protecting group that, if present, is joined to the N-terminal amino group; and Z² is NH2 or an optionally present protecting group that, if present, is joined to the C-terminal carboxy group, and pharmaceutically acceptable salts thereof.

As described in US 2012/0094898 (incorporated herein by reference for the specific recitation of the following compounds), additional NMUR1 agonists include peptide derivatives selected from the group consisting of PEG20k(AL)-β-Ala-Tyr-Nal(1)-Leu-Phe-Arg-Pro-Arg-Asn-NH2, PEG20k(AL)-β-Ala-Tyr-Nal(2)-Leu-Phe-Arg-Pro-Arg-Asn-NH2, PEG20k(AL)-NpipAc-Tyr-Nal(2)-Leu-Phe-Arg-Pro-Arg-Asn-NH2, PEG20k(AL)-NpipAc-Tyr-Nal(2)-Leu-Phe-Arg-Ala-Arg-Asn-NH2. PEG20k(AL)-PEG(2)-Tyr-Nal(2)-Leu-Phe-Arg-NMeAla-Arg-Asn-NH2, PEG20k(AL)-Pic(4)-Tyr-Nal(2)-Leu-Phe-Arg-NMeAla-Arg-Asn-NH2, PEG20k(AL)-Acp-Tyr-Nal(2)-Leu-Phe-Arg-NMeAla-Arg-Asn-NH2, and PEG20k(AL)-β-Ala-Tyr-Nal(2)-Leu-Pya(4)-Arg-Pro-Arg-Asn-NH2, or a salt of any of the peptide derivatives.

As described in WO 2011/005611 (incorporated herein by reference for the specific recitation of the following compounds), additional NMUR1 agonists include compositions comprising the formula

Z¹-peptide-Z²

wherein the peptide has the amino acid sequence X¹-X²-X³-X⁴-X⁵-X⁶-X7-X⁸-X⁹-X¹⁰-X¹¹-X¹²-X¹³-X¹⁴-X¹⁵-X¹⁶-X¹⁷-X¹⁸-X¹⁹-X²⁰-X²¹-X²²-X²³-X²⁴-X²⁵, wherein amino acids 1 to 17 can be any amino acid or absent; wherein amino acid X¹⁸ is absent, Tyr or D-Tyr, Leu, Phe, Val, Gln, Nle, Glu or D-Glu, Asp, Ala, D-Lys, an aromatic amino acid, a des-amino acid or an acyl group; amino acid X¹⁹ is Ala, Trp, Tyr, Phe, Glu, Nva, Nle or an aromatic amino acid; amino acid X²⁰ is absent, Leu, Gly, sarcosine (Sar), D-Leu, NMe-Leu, D-Ala or Ala, or any D- or L-amino acid; amino acid X²¹ is Phe, NMe-Phe, an aliphatic amino acid, an aromatic amino acid, Ala or Trp; X²² is Arg, Lys, Harg, Ala, or Leu; amino acid X²³ is Pro, Ser, Sar, Ala or Leu; amino acid X²⁴ is Arg, Harg or Lys; and amino acid X²⁵ is Asn, any D- or L-amino acid, Nle or D-Nle, D-Ala or Ala; Z¹ is optionally a protecting group that, if present, is joined to the N-terminus amino group; and Z² is NH2 or an optionally present protecting group that, if present, is joined to the C-terminal carboxy group, and pharmaceutically acceptable salts thereof.

As described in WO 2010/138343 (incorporated herein by reference for the specific recitation of the following compounds), additional NMUR1 agonists include compositions comprising a neuromedin U receptor agonist in which neuromedin U or an analog thereof is conjugated to cysteine residue 34 of human serum albumin by a non-maleimido or non-succinimidyl linkage or a pharmaceutically acceptable salt thereof.

As described in WO 2009/042053 (incorporated herein by reference for the specific recitation of the following compounds), additional NMUR1 agonists include a neuromedin U receptor agonist represented by the following formula:

Z¹-peptide-Z²

wherein the peptide has the amino acid sequence ILQRGSGTAAVDFTKKDHTATWGRPFFLFRPRN (SEQ ID NO: 5), wherein the peptide can have one or more insertions or substitutions of the amino acid sequence with an alternative amino acid and wherein the peptide can have one or more deletions of the amino acid sequence; Z¹ is an optionally present protecting group that, if present, is joined to the N-terminal amino group; and Z² is NH2 or an optionally present protecting group that, if present, is joined to the C-terminal carboxy group; and pharmaceutically acceptable salts thereof.

As described in WO 2009/044918 (incorporated herein by reference for the specific recitation of the following compounds), additional NMUR1 agonists include neuromedin U derivatives selected from polypeptides consisting of an amino acid sequence which is bound with a methoxypolyethylene glycol(s) via a linker, wherein the amino acid sequence contains at least 8 amino acids of the C-terminus of an amino acid sequence of neuromedin U, and is the same or substantially the same as the amino acid sequence of neuromedin U.

### Antagonists of Neuromedin U Receptor 1 (NMUR1) or Neuromedin U (NMU)

Antagonists of NMUR1 include peptide antagonists (including modified peptides and conjugates), inhibitory antibody molecules, inhibitory nucleic acid molecules, and small molecules. The NMUR1 antagonists may be entirely specific for NMUR1, may antagonize NMUR1 preferentially (as compared to neuromedin U receptor 2, NMUR2), or may antagonize both NMUR1 and NMUR2. Such antagonists may be useful even if NMUR1 is antagonized less than NMUR2, but it is preferred that the antagonists used in the methods described herein antagonize NMUR1 to a greater extent than NMUR2. As used herein, antagonizing NMUR1 preferentially (as compared to neuromedin U receptor 2, NMUR2) means that the antagonist antagonizes NMUR1 at least 10%, 25%, 50%, 100%, 200%, 300%, 400%, 500%, 600%, 700%, 800%, 900%, 1000%, or more than NMUR2.

As described in US 2011/0165144 (incorporated herein by reference for the specific recitation of the following compounds), additional NMU and NMUR1 antagonists include

(i) a neuromedin U (NMU)-specific inhibitory nucleic acid, e.g., an siRNA, antisense, aptamer, or ribozyme targeted specifically to NMU;

(ii) a neuromedin U (NMU) inhibitory peptide, e.g., a peptide comprising the sequence Phe-Arg-Pro-Arg-Asn (SEQ ID NO: 6); or

(iii) an antibody or antigen binding fragment thereof that binds to an NMU-R, e.g., NMU-R1, and inhibits NMU signalling, e.g., inhibits binding of NMU to the NMU-R1.

Suitable NMUR1 antagonists also can include:

(i) a neuromedin U receptor 1 (NMUR1)-specific inhibitory nucleic acid, e.g., an siRNA, antisense, aptamer, or ribozyme targeted specifically to NMUR1; or Suitable NMU antagonists also can include:

(i) a soluble NMUR1 molecule that binds NMU, such as an extracellular portion of NMUR1 (e.g., amino acids 1-65 of UniProtKB—Q9HB89) optionally linked or fused to another polypeptide sequence for stability or other functions, such as an immunoglobulin Fc region; and

(ii) an antibody or antigen binding fragment thereof that binds to an NMU, e.g., NMU-8, NMU-23, or NMU-25, and inhibits NMU signalling, e.g., inhibits binding of NMU to the NMU-R1.

A subject shall mean a human or vertebrate mammal including but not limited to a dog, cat, horse, goat and non-human primate, e.g., monkey. Preferably the subject is a human. In some embodiments the subject is one who is not otherwise in need of treatment with an NMUR1 agonist or NMUR1 antagonist. Therefore the subject, in specifically identified embodiments, may be one who has not been previously diagnosed with a disorder for which an NMUR1 agonist or NMUR1 antagonist is an identified form of treatment.

The subject can be first identified as a subject in need of treatment, such as one having a disease that is treatable by the methods disclosed herein, and then treated with an NMUR1 agonist (and/or activated ILC2s) or NMUR1 antagonist. The skilled artisan is aware of methods for identifying a subject as having a disease that is treatable by the methods disclosed herein.

As used herein, the terms “treat,” “treated,” or “treating” refers to a treatment of a disease that ameliorates the disease (disease modification), ameliorates symptoms of the disease, prevents the disease from becoming worse, or slows the progression of the disease compared to in the absence of the therapy.

A “disease associated with Group 2 innate lymphoid cells (ILC2s)” as used herein is a disease or disorder in which ILC2s play some role in the development, maintenance or worsening of the disease or disorder.

In some of the methods disclosed herein, such diseases can be effectively treated by increasing activity or proliferation of ILC2s, such as by contacting ILC2s with an agonist of neuromedin U receptor 1 (NMUR1) in an amount effective to increase activity of the ILC2s; by administering to a subject in need of such treatment an agonist of NMUR1 in an amount effective to treat the disease; or by administering activated ILC2s (and optionally an agonist of NMUR1) in an amount effective to treat the disease.

Diseases treatable by such methods include: infection, tissue repair, wound healing, obesity, diseases treatable by increasing induction of type 2 immune responses, diseases treatable by metabolic regulation, diseases treatable by increasing eosinophils, and diseases treatable by increasing mast cells

In other of the method disclosed herein, the diseases can be effectively treated by decreasing activity or proliferation of ILC2s, such as by contacting ILC2s with an antagonist of neuromedin U receptor 1 (NMUR1) in an amount effective to decrease activity of the ILC2s; or by administering to a subject in need of such treatment an antagonist of NMUR1 in an amount effective to treat the disease.

Diseases treatable by such methods include: allergy, allergic asthma, food allergy, eosinophilic esophagitis, atopic dermatitis, fibrosis, allergic rhinitis, allergic rhinosinusitis, chronic obstructive pulmonary disease (COPD), cystic fibrosis, diseases treatable by reducing type 2 immune responses, diseases treatable by reducing eosinophils, or diseases treatable by reducing mast cells.

Toxicity and efficacy of the methods of the present invention can be determined by standard pharmaceutical procedures in cell cultures or experimental animals, e.g., for determining the LD₅₀ (the dose lethal to 50% of the population) or TD₅₀ (the dose toxic to 50% of the population) and the ED₅₀ (the dose therapeutically effective in 50% of the population). The dose ratio between toxic and therapeutic effects is the therapeutic index and it can be expressed as the ratio LD₅₀/ED₅₀ or TD₅₀/ED₅₀. Therapeutic agents that exhibit large therapeutic indices are preferred. While therapeutic agents that exhibit toxic side effects may be used, in such cases it is preferred to use a delivery system that targets such agents to the site of affected tissue in order to minimize potential damage to other cells or tissues and, thereby, reduce side effects.

The data obtained from the cell culture assays and/or animal studies can be used in formulating a range of dosage of the therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Other, higher percentages of an active compound also can be used.

The pharmaceutical compositions may also be, and preferably are, sterile in some embodiments. In other embodiments the compounds may be isolated. As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane components). In the case of nucleic acid molecules, an isolated nucleic acid includes a PCR product, an isolated RNA, a synthetically (e.g., chemically) produced RNA, such as an siRNA, an antisense nucleic acid, an aptamer, etc. Isolated nucleic acid molecules include sequences inserted into plasmids, cosmids, or other vectors to form part of a chimeric recombinant nucleic acid construct, or produced by expression of a nucleic acid encoding it. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein, or may be synthetically (e.g., chemically) produced, or produced by expression of a nucleic acid encoding it. An isolated cell, such as an ILC2 cell, can be removed from the anatomical site in which it is found in an organism, or may be produced by in vitro expansion of an isolated cell or cell population. An isolated material may be, but need not be, purified.

The term “purified” in reference to a protein, a nucleic acid, or a cell or cell population, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified agonist of NMUR1 or antagonist of NMUR1 or ILC2 population is at least 60%, at least 80%, or at least 90% of total protein or nucleic acid or cell population, as the case may be, by weight. In a specific embodiment, a purified agonist of NMUR1 or antagonist of NMUR1 or ILC2 population is purified to homogeneity as assayed by standard, relevant laboratory protocols.

In some embodiments a purified and or isolated molecule is a synthetic molecule.

Subject doses of the compounds described herein typically range from about 0.1 μg to 10,000 mg, more typically from about 1 μg/day to 8000 mg, and most typically from about 10 μg to 100 Gig. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 1 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment. Multiple doses of the molecules of the invention are also contemplated.

The compounds and/or cells described herein may be used alone without other active therapeutics or may be combined with other therapeutic compounds for the treatment of the diseases described herein.

When used in combination with the compounds and cells described herein, the dosages of known therapies may be reduced in some instances, to avoid side effects. In some instances, when the compounds and/or cells described herein are administered with another therapeutic, a sub-therapeutic dosage of either the compounds and/or cells described herein or the known therapies, or a sub-therapeutic dosage of both, is used in the treatment of a subject. A “sub-therapeutic dose” as used herein refers to a dosage which is less than that dosage which would produce a therapeutic result in the subject if administered in the absence of the other agent. Thus, the sub-therapeutic dose of a known therapy is one which would not produce the desired therapeutic result in the subject in the absence of the administration of the compounds and cells described herein. Existing therapies for the diseases described herein are well known in the field of medicine, and may be described in references such as Remington's Pharmaceutical Sciences; as well as many other medical references relied upon by the medical profession as guidance for treatment.

When the compounds and/or cells described herein are administered in combination with other therapeutic agents, such administration may be simultaneous or sequential. When the other therapeutic agents are administered simultaneously they can be administered in the same or separate formulations, but are administered at the same time. The administration of the other therapeutic agent and the compounds and/or cells described herein can also be temporally separated, meaning that the other therapeutic agents are administered at a different time, either before or after, the administration of the compounds and cells described herein. The separation in time between the administration of these compounds may be a matter of minutes or it may be longer.

The active agents of the invention (e.g., the compounds and cells described herein) are administered to the subject in an effective amount for treating disease. According to some aspects of the invention, an effective amount is that amount, depending on the disease being treated, of a NMUR1 agonist (and/or activated ILC2s) or NMUR1 antagonist alone or in combination with another medicament, which when combined or co-administered or administered alone, results in a therapeutic response to the disease. The biological effect may be the amelioration and or absolute elimination of disease, or of symptoms resulting from the disease. In another embodiment, the biological effect is the complete abrogation of the disease, as evidenced for example, by the absence of a symptom of the disease.

The effective amount of a compound (i.e., any of the agonists, antagonists, or ILC2s) used in methods of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention using routine and accepted methods known in the art, without necessitating undue experimentation. Combined with the teachings provided herein, by choosing among the various active compounds and weighing factors such as potency, relative bioavailability, patient body weight, severity of adverse side-effects and preferred mode of administration, an effective therapeutic treatment regimen can be planned which does not cause substantial toxicity and yet is effective to treat the particular subject.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by relevant government regulatory agencies. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The therapeutic compositions used as described herein may comprise different types of carriers depending on whether it is to be administered in solid, liquid or aerosol form, and whether it need to be sterile for such routes of administration as injection. The compounds and/or cells described herein can be administered intravenously, intradermally, intraarterially, intralesionally, intracranially, intraarticularly, intranasally, intravitreally, intravaginally, intrarectally, topically, intramuscularly, intraperitoneally, subcutaneously, intravesicularlly, mucosally, orally, locally, by inhalation (e.g., aerosol inhalation), by injection, by infusion including by continuous infusion, by localized perfusion, via a catheter, via a lavage, in cremes, in lipid compositions (e.g., liposomes), or by other method or any combination of the foregoing as would be known to one of ordinary skill in the art (see, for example, Remington's Pharmaceutical Sciences) and as is appropriate for the disease being treated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds described herein may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the compounds and/or cells described herein is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The compounds and/or cells described herein can be administered in various ways and to different classes of recipients. In some instances the administration is chronic. Chronic administration refers to long term administration of a drug to treat a disease. The chronic administration may be on an as needed basis or it may be at regularly scheduled intervals. For instance, the compounds and/or cells described herein may be administered twice daily, three times per day, four times per day, every other day, weekly, every two weeks, every four weeks, continuously (e.g., by infusion, patch, or pump), and so on.

The compounds and/or cells described herein may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The compounds and/or cells described herein are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods described herein, the compounds and/or cells described herein may be administered in a pharmaceutical composition. In general, a pharmaceutical composition comprises the compound of the invention and a pharmaceutically-acceptable carrier. Pharmaceutically-acceptable carriers useful with compounds and/or cells described herein are well-known to those of ordinary skill in the art. As used herein, a pharmaceutically-acceptable carrier means a non-toxic material that does not interfere with the effectiveness of the biological activity of the compounds and/or cells described herein.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds and/or cells described herein may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds and/or cells described herein may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Remington's Pharmaceutical Sciences). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, vehicle for the compounds and/or cells described herein is a biocompatible microparticle or implant that is suitable for implantation into a mammalian recipient. Exemplary bioerodible implants are known in the art. The implant may be a polymeric matrix in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the compounds and/or cells described herein to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the compounds and/or cells described herein may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Such delivery systems also include non-polymer systems such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. In addition, pump-based hardware delivery systems can be used, some of which are adapted for implantation.

Use of a long-term sustained release implant may be particularly suitable for treatment of chronic diseases. Long-term release, as used herein, means that the implant is constructed and arranged to delivery therapeutic levels of the active ingredient for at least 30 days, and preferably at least 60 days. Long-term sustained release implants are well-known to those of ordinary skill in the art and include some of the systems described above.

Thus the compounds and/or cells described herein described herein may, in some embodiments, be assembled into pharmaceutical or research kits to facilitate their use in therapeutic or research applications. A kit may include one or more containers housing the components of the invention and instructions for use. Specifically, such kits may include one or more compounds and/or cells described herein, along with instructions describing the intended therapeutic application and the proper administration of these agents. In certain embodiments the compounds and/or cells described herein in a kit may be in a pharmaceutical formulation and dosage suitable for a particular application and for a method of administration of the agents.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Materials and Methods

Mice: C57BL/6J (B6) mice were purchased from Charles River. Nod/Scid/Gamma (NSG) mice were bought from the Jackson Laboratory. Sperm from the strain C57BL/6N-Nmur1^{tm1.1(KOMP)Vlcg}, which contains a Nmur1 deletion, was obtained from the KOMP Repository, located at the University of California Davis and Children's Hospital Oakland Research Institute, US. Nmur1^{-/-} mice were generated by in vitro fertilization at the Champalimaud Centre for the Unknown, Portugal. Ret^{GFP 16} mice were on a C57Bl/6J background. Mice were bred and maintained at the iMM Lisboa animal facility under specific pathogen free conditions. Mice were systematically compared with co-housed littermate controls. Both males and females were used in this study. All animal experiments were approved by national and institutional ethical committees, respectively Diregio Geral de Veteriniria and iMM Lisboa ethical committee. Randomization and blinding were not used unless stated otherwise. Power analysis was performed to estimate the number of experimental mice.

Analysis of Gene Expression Microarray Data:

The expression profile of 239 genes related to neural pathways was performed in mouse lymphoid cells based on the Affymetrix Mouse Gene 1.0 ST Array dataset (GEO accession number GSE37448)¹⁰. Preprocessing of microarray data (including background correction and normalization) was performed applying the robust multiarray (RMA) method³⁰, included in the Bioconductor package affy³¹ for the statistical software environment R³². Linear models and the B (empirical Bayes) statistic were employed in differential gene expression analysis, using Bioconductor package limma³³. Plots associated with the microarray data analyses were generated in R.

Bone Marrow Transplantation:

Bone marrow cells were flushed out from femurs and tibiae of Nmur1^{-/-} and WT littermate controls. Bone marrow cells were CD3-depleted using Dynabeads Biotin Binder (Thermo Fisher Scientific) according to the manufacturer's instructions. 10⁶ cells of each genotype (CD45.2) were injected intravenously in direct competition with a third-party WT competitor (CD45.1/CD45.2), in a 1:1 ratio, into non-lethally irradiated (150 Rad) NSG mice (CD45.1). Mice were analysed at 8 weeks after transplantation.

In Vitro and In Vivo Neuromedin U Activation:

For in vitro experiments, purified lung and small intestine lamina propria ILC2s were FBS starved for 2 hours prior to stimulation and cultured in complete RPMI (supplemented with 10% foetal bovine serum (FBS), 1% hepes, sodium pyruvate, glutamine, streptomycin and penicillin) at 37° C. For mRNA analysis, ILC2s were stimulated overnight with recombinant mouse Neuromedin U 23 peptide (NmU23, 100 ng/mL; Phoenix pharmaceuticals). Both NmU23-stimulated and control ILC2s were cultured in the presence of IL-2 and IL-7 (10 ng/mL; Peprotech). ILC2s were lysed using RLT buffer (Qiagen). For cytokine protein analysis, ILC2s were incubated exclusively with brefeldin A (eBioscience) for 12 hours prior to intracellular staining. For in vivo experiments, B6 mice were injected intraperitoneal with NmU23 peptide (2 g/day) during Nippostrongylus brasiliensis infection or with a single dose of NmU23 (20 μg) and analyzed after 8 hours. Control mice were treated with PBS alone.

Parasite Infection:

Nippostrongylus brasiliensis was maintained by monthly passages in Lewis rats as previously described³⁴. Infective (iL3) worms were kindly provided by Nicola Harris (Lausanne, Switzerland). iL3 larvae were treated for 15 minutes with penicillin/streptomycin (300 U/mL; Thermo Fisher Scientific), gentamicin (1.5 mg/mL; Sigma) and tetracyclin (30 μg/mL; Sigma), washed with PBS and counted under a stereomicroscope. Mice were injected subcutaneously with 500 iL3 in 200 μL of sterile PBS using a 21 G needle. Mice were sacrificed at day 2 post-infection and lungs were collected and analysed.

Burden of Infection:

Lung parasite burden was quantified in finely minced lungs and as previously described³⁴. Lung were placed on sterile cheesecloth and suspended in a 50 mL tube containing PBS at 37° C. for at least 4 hours. Viable worms that migrate out into the bottom of the tube were counted under a stereomicroscope (steREO Lumar V12; Zeiss).

Cell Isolation:

Lungs were perfused with a solution of cold PBS and 2% heparin through the right ventricle of the heart and were subsequently finely minced and digested in complete RPMI supplemented with collagenase D (0.1 mg/mL; Roche) and DNase I (20 U/mL; Affymetrix) for 1 h at 37° C. under gentle agitation. For isolation of small intestine lamina propria cells, intestines were thoroughly rinsed with PBS, cut in 1 cm pieces, and shaken for 30 minutes in PBS containing 2% FBS, 1% hepes and 5 mM EDTA to remove intraepithelial and epithelial cells. Intestines were then digested with collagenase D (0.5 mg/mL; Roche) and DNase I (20 U/mL; Affymetrix) in complete RPMI for 30 minutes at 37° C., under gentle agitation. Enteric neurons and glial cells were isolated as previously described^{4,35}. Briefly, isolated tissues were digested with Liberase™ (7,5 μg/mL; Roche) and DNase I (20 U/mL; Affymetrix) in complete RPMI for 30 minutes at 37° C., under gentle agitation. Digested organs were disrupted by passage through a 100 μm cell strainer (BD Biosciences). A 40-80% percoll gradient centrifugation (2,400 rpm, 30 minutes at room temperature) was used for additional leukocyte purification from lung and small intestine cell suspensions. Erythrocytes from lung, small intestine and bone marrow preparations were lysed with RBC lysis buffer (eBioscience).

Flow Cytometry and Cell Sorting:

Intracellular staining was performed using IC fixation/permeabilization kit (eBioscience). Flow cytometry analysis and cell sorting were performed using BD LSRFORTESSA and BD FACSAria flow cytometers (BD Biosciences). Data analysis was done using FlowJo software (Tristar). Sorted populations were >95% pure. Cell suspensions were stained with anti-CD45 (30-F11), anti-TER119 (TER-119), TCRPβ (H57-597), anti-CD3ε (eBio500A2), anti-CD19 (eBio1D3), anti-NK1.1 (PK136), anti-CD11c (N418), anti-Gr1 (RB6-8C5), anti-CD11b (Mi/70), anti-CCR6 (29-2L17), anti-CD127 (IL-7Ra; A7R34), anti-α4β7 (DATK32), anti-Ft3 (A2F10), anti-CD25 (PC61.5), anti-cKit (2B8), anti-Thy1.2 (53-2.1), anti-CD49b (DX5), anti-CD49a (HMα1), anti-TCRδ (GL3), anti-Nkp46 (29A1.4), anti-CD4 (GK1.5), anti-CD31 (390), anti-IL-13 (eBio13A), anti-IL-4 (AAB11), anti-CSF2 (MP1-22E9), anti-F4/80 (BM8), anti-FεR1 (MAR-1), 7AAD viability dye, Anti-Mouse CD16/CD32 (Fc block) all from eBioscience; anti-CD8α (53-6.7), anti-KLRG1 (2F1), anti-sca1 (D7), anti-CCR3 (J073E), anti-MHC-II (M5/114.15.2) from biolegend, anti-IL-5 (MH9A3) from BD Biosciences, anti-amphiregulin (R&D). LIVE/DEAD Fixable Aqua Dead Cell Stain Kit was purchased from Invitrogen. Cell populations were defined as: ILC2—CD45⁺Lin⁻Thy1.2⁺KLRG1⁺Sca1⁺; ILC3—CD45⁺Lin⁻ Thy1.2^{hi}IL7Rα⁺RORγt⁺; for ILC3 subsets additional markers were employed: LTi—CCR6⁺Nkp46⁻; ILC3 NCR⁻—CCR6⁻Nkp46⁻; ILC3 NCR⁺—CCR6⁻Nkp46⁺; NK cells—CD45⁺Lin⁻NKp46⁺NK1.1⁺CD49b⁺CD49a⁻CD127⁻; Lineage was composed by CD3ε, CD8α, TCRβ, TCRγδ, CD19, Gr1, CD11c and TER119; enteric glial cells—CD45⁻CD31⁻TER119⁻ CD49b⁺; T cells—CD45⁺CD3⁺TCRβ⁺; B cells—CD45⁺CD19⁺; enteric neurons—CD45⁻ CD31⁻TER119⁻RET⁺; Eosinophils—MHC-II⁻CCR3^{hi}GR1^{int}; Neutrophils—MHC-II⁻CCR3⁻ GR1^{hi}; Macrophages—CD3⁻MHC-II⁺F4/80⁺; Mastocytes/Basophils—CD3⁻FcεR1⁺; Common Lymphoid Progenitor (CLP)—Lin⁻CD127⁺Flt3⁺Sca1^{int}cKit^{int}; Common Helper Innate Lymphoid Progenitor (CHILP)—Lin⁻CD127⁺α4β7⁺Flt3⁻CD25⁻; ILC2 precursor (ILC2P)—Lin⁻CD127⁺α4β7⁺Flt3⁻CD25⁺.

Quantitative RT-PCR:

Total RNA was extracted using RNeasy micro kit (Qiagen) according to the manufacturer's protocol. RNA concentration was determined using Nanodrop Spectrophotometer (Nanodrop Technologies). Quantitative real-time RT-PCR was performed as previously described^{5,8}. Hprt1, Gapdh and Eef1a1 were used as housekeeping genes. For TaqMan assays (Applied Biosystems) RNA was retro-transcribed using a High Capacity RNA-to-cDNA Kit (Applied Biosystems), followed by a pre-amplification PCR using TaqMan PreAmp Master Mix (Applied Biosystems). TaqMan Gene Expression Master Mix (Applied Biosystems) was used in real-time PCR. TaqMan Gene Expression Assays (Applied Biosystems) were the following: Hprt1 Mm00446968_m1; Gapdh Mm99999915_g 1; Eef1a1 Mm01973893_g 1; Il5 Mm00439646_m1; Il13 Mm00434204_m1; Areg Mm01354339_m1; 114 Mm00445259_m1; Csf2 Mm01290062_m1; Gata3 Mm00484683_m1; Rora Mm01173766_m1; Nmu Mm00479868_m1; Nmur1 Mm04207994_m1. Real-time PCR analysis was performed using StepOne Real-Time PCR system (Applied Biosystems).

Cell Signalling:

Purified ILC2s from small intestine and lung were FBS starved for 2 hours before in vitro activation with NmU23 at 37° C. To test for ERK phosphorylation (Cell Signaling Technology), purified ILC2s were activated with NmU23 (100 ng/mL; Phoenix pharmaceuticals) in the presence of IL-2 and IL-7 (10 ng/mL; Peprotech) for 10 minutes prior to intracellular staining. To test ERK, calcineurin and NFAT activation, ILC2s were cultured for 1 hour with their respective inhibitor and then stimulated with NmU23 overnight before mRNA expression analysis. ERK inhibitor—PD98059 (Sigma); Calcineurin inhibitor—FK506 (Tocris Bioscience); NFAT inhibitor—11R-VIVIT (Tocris Bioscience).

Calcium Signaling:

Purified ILC2s from the small intestine were cultured with IL-2 and IL-7 (10 ng/mL) and FBS deprived for 6 hours prior to calcium signaling experiments. ILC2s were stained with Fluo-4 Direct Calcium Assay Kit (Thermo Fisher Scientific) according to manufacturer's protocol. Calcium (Ca²⁺) influx, represented by the Fluo-4 AM, was recorded over time on a BD Accuri C6 (BD Biosciences) flow cytometer as previously reported³⁶. The recombinant mouse NmU23 was added 60 seconds after ILC2 baseline recording. Data was represented by the mean values of Ca²⁺ influx kinetics between the ILC2 baseline response and the peak of response after recombinant mouse NmU23 addition.

Histopathology Analysis:

Mice were sacrificed by cervical dislocation, and caudal lobe of the right lung was harvested, fixed in 10% neutral buffered formalin and processed for paraffin embedding. Serial 4 μm sections were stained for hematoxylin and eosin (H&E), Luna stain, and immunohistochemistry for myeloperoxidase (MPO) was performed. Briefly, using standard protocols, antigen heat-retrieval was performed at low pH³⁷ in Dako PT module, followed by incubation with the primary antibody (polyclonal rabbit anti-human Myeloperoxidase, Dako Corp). Incubation with ENVISION kit (Peroxidase/DAB detection system, Dako Corp) was followed by Harri's hematoxylin counterstaining (Bio Otica). Negative control included the absence of primary antibodies. Slides were analyzed by a pathologist blinded to experimental groups and images were acquired in a Leica DM2500 microscope, coupled with a Leica MC 170 HD microscope camera. Quantification of inflammatory cell infiltration of the lung was performed in MPO-stained sections by manual counting of MPO-positive cells at 20× original magnification, corresponding to 0.2 mm² per field. Quantification of pulmonary eosinophils was performed in Luna-stained slides by manual counting the number of granulocytes with eosinophilic granular cytoplasm in low power fields (1 mm² per field).

Microscopy:

Analysis of thick gut sections intestines were fixed with 4% PFA at 4° C. overnight and were then included in 4% low-melting temperature agarose (Invitrogen). Sections of 100 μm were obtained with a Leica VT1200/VT1200 S vibratome. Sections were incubated overnight or for 1-2 days respectively at 4° C. using the following antibodies: mouse monoclonal anti-KLRG1 (2F1/KLRG1; Biolegend); anti-CD3 (17A2; Biolegend). A647 goat anti-hamster and A568 goat anti-rat were purchased from Invitrogen. After several washing steps with PBS samples were incubated with antibodies during 3 hour at room temperature and then mounted in Mowiol⁵. Samples were acquired on a Zeiss LSM710 confocal microscope using EC Plan-Neofluar 10×/0.30 M27, Plan Apochromat 20×/0.8 M27 and EC Plan-Neofluar 40×/1.30 objectives.

statistics: results are shown as mean±sem. statistical analysis was performed with GraphPad Prism software (GraphPad Software, La Jolla, Calif). Student's t-test was performed on homocedastic populations. Unpaired t-test was applied on samples with different variances. Results were considered significant at *P<0.05, **P<0.01, ***P<0.001, ****P<0.0001.

### Example 1: Expressions of Neuromedin U Receptor 1 (Nmur1) in ILC2s

Group 2 innate lymphoid cells (ILC2s) are abundant at mucosal barriers and act as key initiators of type 2 inflammation and tissue repair^{1,2}. ILC2s are activated by cell-extrinsic cytokines, including IL-25, IL-33 and thymic stromal lymphopoietin^{1,2}. Previous reports indicated that discrete lymphocyte subsets and haematopoietic progenitors are controlled by dietary signals and neuroregulators^{2,3,5-9}, suggesting that ILC2s may exert their function in the context of neuro-immune cell units.

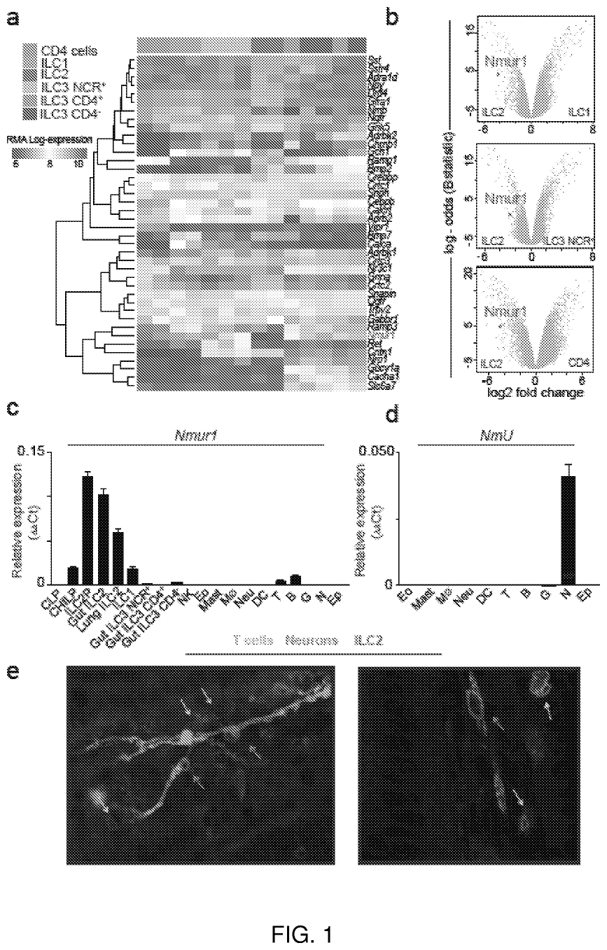

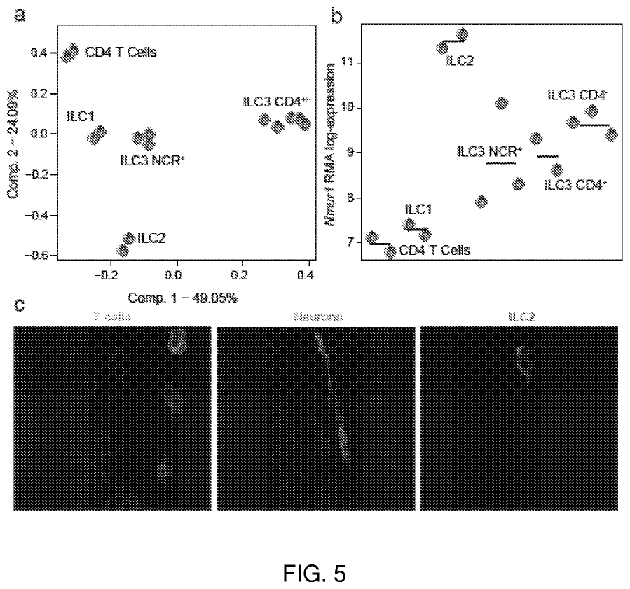

To interrogate whether ILC2s directly and selectively perceive neuronal-derived molecules, genome-wide transcriptional profiling of ILC2s versus their adaptive (T helper lymphocytes) and innate (ILC1 and ILC3) counterparts¹⁰ was employed (FIG. 1a-1b). This analysis identified the gene neuromedin U receptor 1 (Nmur1) as being selectively enriched in ILC2s when compared to ILC1s, ILC3s and T helper 2 cells (FIGS. 1a-1b and FIGS. 5a,5b). This finding was confirmed by independent quantitative expression assays in multiple subsets of immune cells, including ILC1s, ILC3s, NK cells, eosinophils, mast cells, macrophages, neutrophils, dendritic cells, T cells and B cells (FIG. 1c).

Nmur1 encodes for a transmembrane receptor for Neuromedin U. The latter is a secreted neuropeptide found in the brain and highly expressed in the gastrointestinal tract¹¹⁻¹⁴ As such, Neuromedin U (NMU) acts as a neuronal-derived regulator in diverse physiologic processes¹⁴. Neuromedin U was shown to be produced by enteric neurons, which also express the neurotrophic factor receptor RET^{11-13,15}. In agreement, neurons in the lamina propria were main expressers of the Neuromedin U gene (Nmu), while these transcripts were not detectable in enteric neuroglia and epithelial cells (FIG. 1d). Similarly, all analysed immune cell subsets, including dendritic cells, macrophages and B cells, had no significant Nmu expression (FIG. 1d). Strikingly, reporter mice for enteric neurons (Ret^{GFP})¹⁶ revealed that lamina propria CD3⁻KLRG1⁺ candidate ILC2s are adjacent to the intestinal lamina propria Ret^{GFP} neuronal network (FIG. 1e and FIG. 5c). Taken together these data suggest a paracrine neuron-ILC2 crosstalk orchestrated by NMU-NMUR1 interactions.

### Example 2: Activation of ILC2s with Neuromedin U

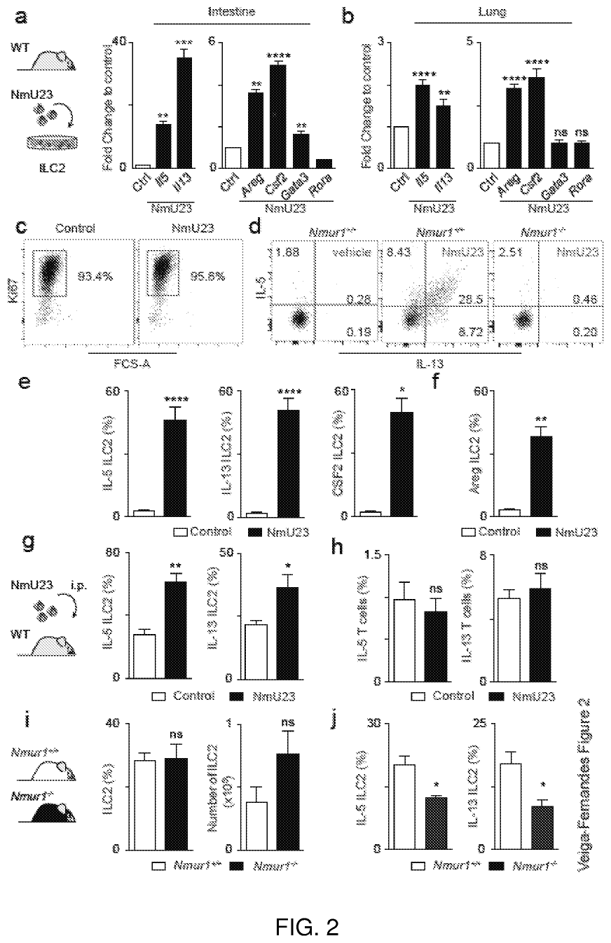

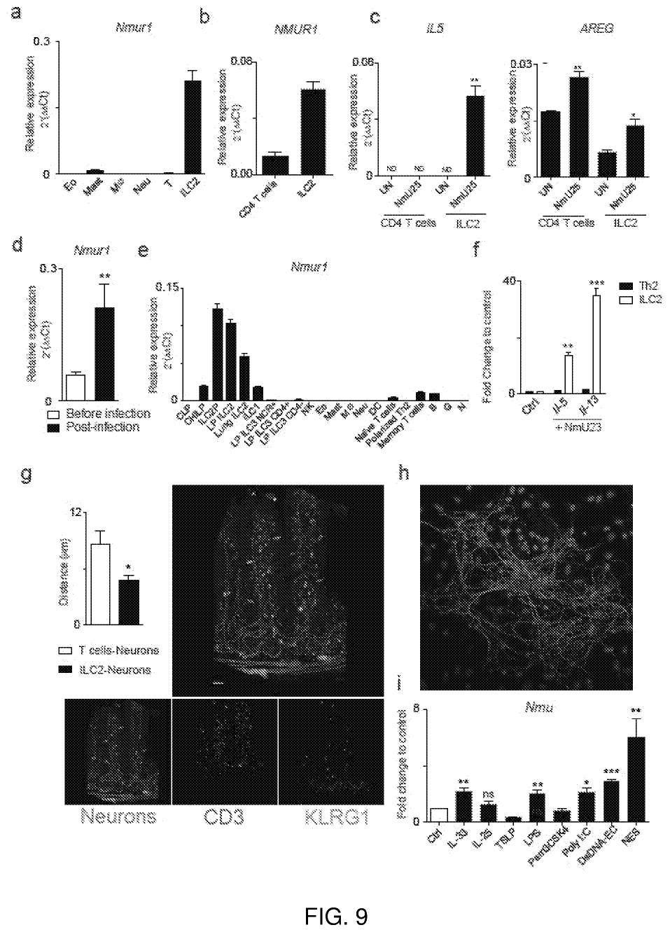

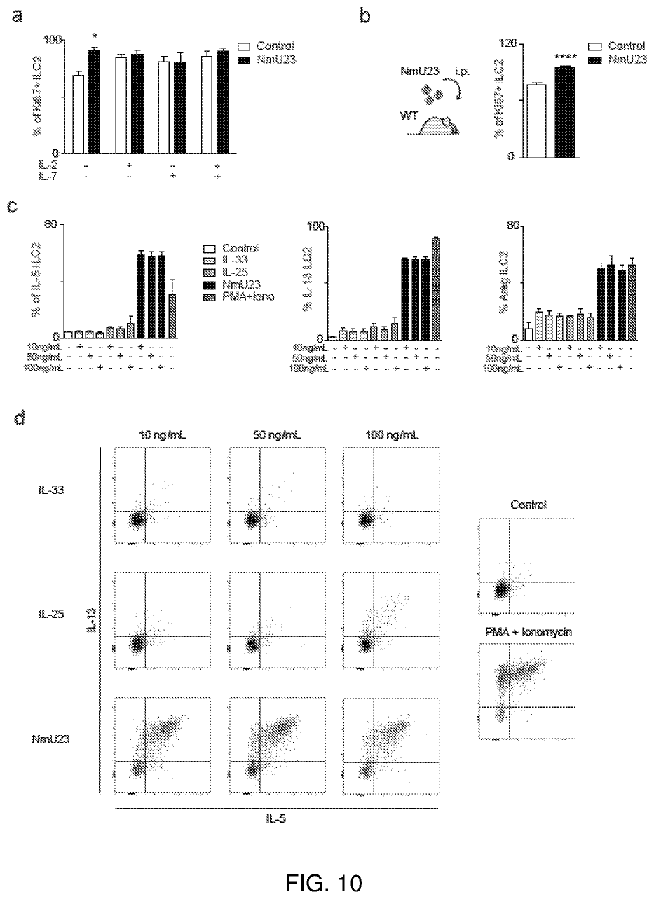

To explore this hypothesis, intestine and lung-derived ILC2s were purified and activated with Neuromedin U (NmU23 neuropeptide) (FIGS. 2a-2f). Astonishingly, cell-autonomous activation of ILC2s with NmU23 resulted in prompt and very potent expression of the pro-inflammatory and tissue-protective type 2 cytokines genes Il5, Il13, Areg and Csf2, which was paralleled by increased expression of the master type 2 transcription factor Gata3 (FIGS. 2a,2b). Similar finding were obtained with human ILC2 (FIG. 9b,c). NmU23-dependent activation of ILC2s increased ILC2 proliferation as measured by Ki67 (FIG. 2c; FIG. 10a, 10b). NmU was shown to bind with similar affinity to two orphan class A G-protein-coupled receptors, NMUR1 and NMUR2¹⁴.

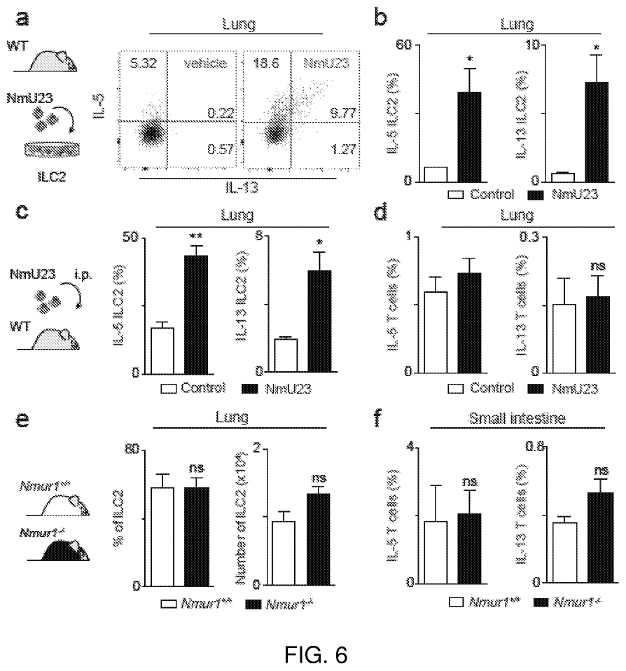

Formal definition that NMUR1 activation is the molecular link between NMU-dependent ILC2 activation and type cytokine production was provided by genetic ablation of Nmur1. Activation of purified ILC2s with NmU23 led to potent expression of the type 2 cytokine proteins IL-5 and IL-13 in a NMUR1-dependent manner (FIG. 2d-2f and FIG. 6a,6b).

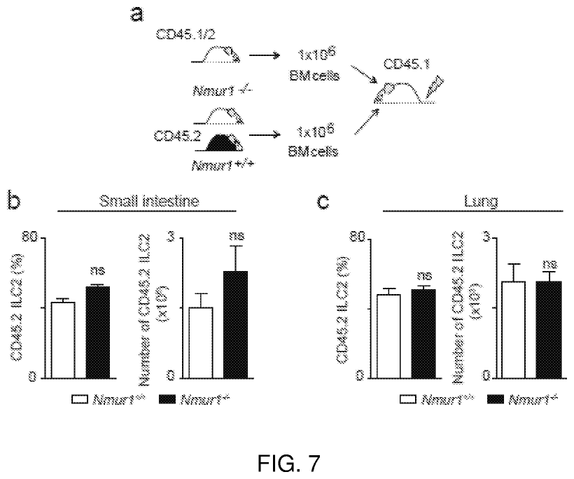

Importantly, in vivo administration of the neuropeptide NmU23 resulted in immediate and selective type 2 cytokine production from ILC2s, while their adaptive T helper cell-derived counterparts were unperturbed (FIG. 2g,2h and FIG. 6c,6d). In agreement, Nmur1 deficient mice had an intact ILC2 compartment, but reduced innate IL-5 and IL-13 expression when compared to their wild-type (WT) littermate controls (FIG. 2i,2j; FIG. 6e,6f; and FIG. 7a-7c). It was noteworthy that T helper cell-derived cytokines were unperturbed in Nmur1 knockout mice (FIG. 6f).

These data indicate that the neuropeptide Neuromedin U is a potent regulator of innate type 2 inflammatory and tissue repair cytokines, via NMUR1 activation.

### Example 3: Signalling by Activated NMUR1 in ILC2s

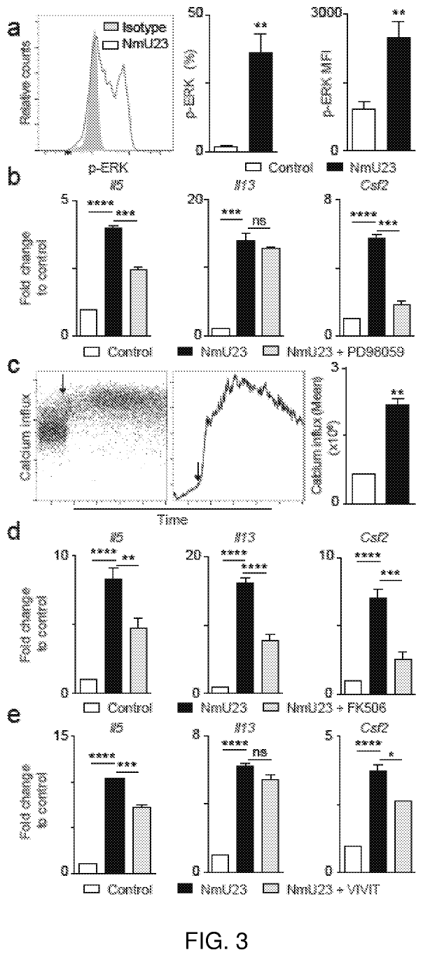

To further examine how Neuromedin U controls innate type 2 responses the signalling cues provided by activated NMUR1 in ILC2s were investigated. In neurons activation of Neuromedin U receptors leads to increased Calcium (Ca²⁺) influx and ERK1/2 activation, while NFAT activity is required for type 2 cytokine production¹⁷⁻²⁰. Neuromedin U-induced activation of ILC2s led to immediate and efficient ERK1/2 activation, while inhibition of ERK activity upon NmU23-induced ILC2 activation resulted in impaired type 2 cytokine gene expression (FIG. 3a,3b).

Analysis of Neuromedin U-induced activation of ILC2s also led to immediate and robust Ca²⁺ influx, suggesting a role of the calcium dependent serine/threonine protein phosphatase Calcineurin in NmU23-induced type 2 responses (FIG. 3c). In agreement, inhibition of Calcineurin upon NmU23 activation led to impaired innate Il5, Il13 and Csf2 expression (FIG. 3d).

Finally, inhibition of NFAT activity upon NmU23-induced NMUR1 activation led to similarly decreased Il5, Il13 and Csf2 (FIG. 3e). Thus, it was concluded that the neuronal-derived peptide Neuromedin U can operate in an ILC2-intrinsic manner by activating NMUR1, which regulates innate type 2 cytokines downstream of a Ca²⁺/Calcineurin/NFAT cascade and ERK1/2 phosphorylation.

### Example 4: Regulation of Mucosal Defence by ILC2 Cells

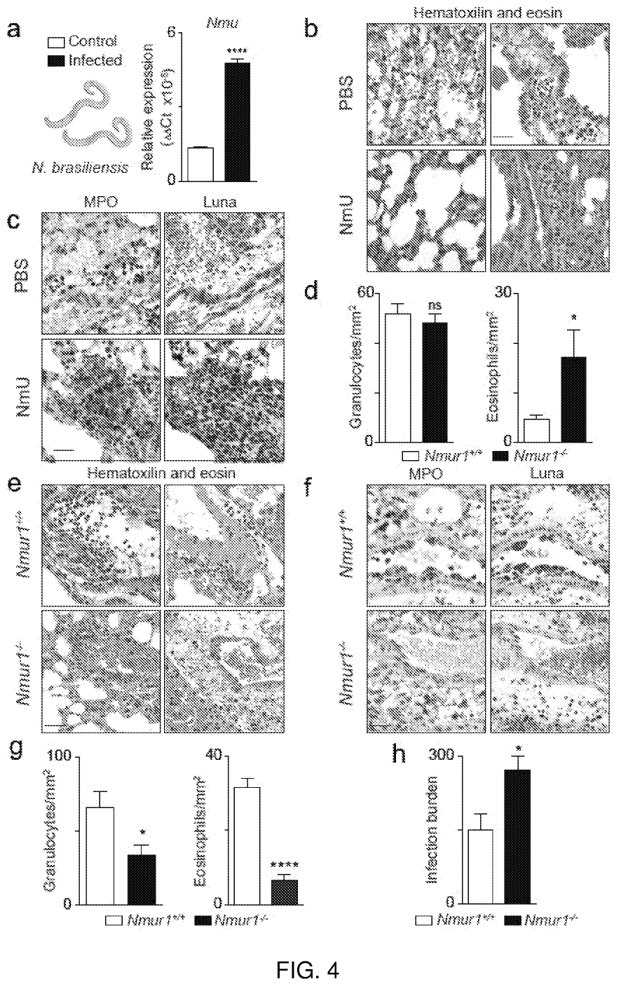

To interrogate whether neuronal peptides regulate mucosal defence, how varying degrees of NMUR1 signals can control mucosal aggression shortly after infection with the helminthic parasite Nippostrongylus brasiliensis²¹, and before adaptive T cell responses are established²² was tested. Strikingly, infection of WT mice with N. brasiliensis resulted in strongly increased Nmu expression in the lung (FIG. 4a), suggesting that Neuromedin U may regulate in vivo responses to worm infection. Accordingly, administration of the neuropeptide NmU23 in N. brasiliensis infected mice resulted in very robust and immediate innate type 2 responses characterised by increased eosinophil infiltrates in the lung when compared to their vehicle (PBS) treated counterparts (FIG. 4b-4d).

To further explore the role of NMUR1 in innate type 2 responses, Nmur1 deficient mice and their littermate controls were infected with N. brasiliensis (FIG. 4e-4i). Strikingly, when compared to their WT littermate counterparts, Nmur1 knockout mice had decreased type 2 responses, notably markedly reduced eosinophil and granulocyte infiltrates (FIG. 4e-4g). In line with these findings, Nmur1 deficient mice had increased N. brasiliensis infection burden (FIG. 4i). Altogether, these data indicate that the neuropeptide Neuromedin U provides critical cues that regulate type 2 responses in vivo, thus increasing immediate mucosal protection against worm infections.

### Example 5: Signal Integration by ILC2 Cells

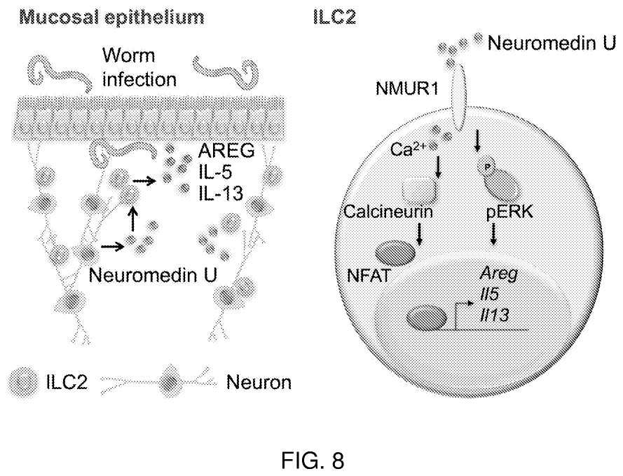

Deciphering the mechanisms by which ILC2s perceive, integrate and respond to environmental signals is critical to understand tissue and organ homeostasis. The results reported herein establish unexpected relationships between ILC2s and their environment. A novel neuron-ILC2 cell unit orchestrated by Neuromedin U was deciphered (FIG. 8). This neuropeptide directly activates ILC2s in a NMUR1 dependent manner, resulting in a potent innate type 2 cytokine production downstream of ERK phosphorylation and activation of a Ca²⁺/Calcineurin/NFAT cascade (FIG. 8).

While it is well-established that ILC2s integrate cytokine signals, including IL-25, IL-33 and thymic stromal lymphopoietin^{1,2,23}, the results reported herein demonstrate that ILC2s can more broadly integrate signals from different germ-layer-derived tissues to simultaneously regulate inflammatory and tissue repair type 2 responses and organ defence. Thus, it is proposed that neuron-ILC2 cell units are poised to uniquely ensure potent and immediate type 2 responses in a neuromedin U-dependent manner (FIG. 8).

Previous studies demonstrated that ILC2s contribute to multiple homeostatic processes, including nutrient sensing, metabolism, tissue repair and infection control^{1,2,21,23-27}. Here it has been shown that neuromedin U is the molecular link between neuronal activity, innate type 2 responses and mucosal protection. Thus, coupling neuronal activity and ILC2-dependent immune regulation may have ensured potent, efficient and integrated multi-tissue responses to environmental challenges throughout evolution. Notably, coordinated, neuromedin U-dependent smooth muscle contraction¹⁴ and type 2 innate immunity may have coevolved to control worms that have been intimate evolution partners of mammals. In line with this hypothesis, neuromedin U is highly conserved across mammalian, amphibian, avian and fish species¹⁴. Finally, the current data and other independent studies indicate that the mucosal nervous system partners with ILCs and macrophages to ensure local tissue regulation^{3,4,28,29}; thus it is tempting to speculate the existence of neuroimmune sensory units that regulate physiology and homeostasis at an organismic level.

### Example 6: Selective Expression of Nmur1 and Activation of ILC2s

Transcriptional analysis identified the gene neuromedin U receptor 1 (Nmur1) as being selectively enriched in ILC2s when compared to ILC1s, ILC3s and T helper 2 cells (FIGS. 1a,1b and FIGS. 5a,5b). This finding was confirmed by independent quantitative expression assays in multiple subsets of immune cells, including ILC1s, ILC3s, NK cells, eosinophils, mast cells, macrophages, neutrophils, dendritic cells, T cells and B cells (FIG. 9e). In line with this finding, activation of ILC2 with NMU23, resulted in immediate innate Il5 and Il13 upregulation, while their adaptive T cell counterparts were unperturbed (FIG. 9f). Noteworthy, after infection with Nippostrongylus brasiliensis Nmur1 expression was selectively increased in ILC2 (FIGS. 9a,9d).

Neurons in the lamina propria were found to be the main expressers of the Neuromedin U gene (Nmu), while these transcripts were not detectable in enteric neuroglia and epithelial cells (FIG. 1d). Similarly, all analysed immune cell subsets, including eosinophils, dendritic cells, macrophages, B cells and T cells, had no significant Nmu expression (FIG. 1d). Strikingly, reporter mice for enteric neurons (Ret^{GFP}) revealed that lamina propria CD3⁻KLRG1⁺ candidate ILC2s found at 4.716 μm±0.656 from adjacent neurons while their adaptive T cell counterparts are found at a significantly bigger distance (8.623 μm±1.447) (FIG. 1e; FIG. 5c; and FIG. 9g). Astonishingly, neurospherere-derived neurons stimulated with N. brasiliensis/secretory proteins (NES) rapidly up-regulated Nmu expression (FIG. 9h,i), indicating that neurons can directly sense worm products to regulate NMU production.

### Example 7: Type 2 Cytokines Expressed Upon Activation of ILC2s

Intestine and lung-derived ILC2s were purified and activated with Neuromedin U (NmU23 neuropeptide) (FIGS. 2a-2f). Astonishingly, cell-autonomous activation of ILC2s with NmU23 resulted in prompt and very potent expression of the pro-inflammatory and tissue-protective type 2 cytokines genes Il5, Il13, Areg and Csf2, which was paralleled by increased expression of the master type 2 transcription factor Gata3 (FIGS. 2a,2b). NmU23-dependent activation of ILC2s increased ILC2 proliferation as measured by Ki67 in vitro and in vivo (FIG. 2c and FIGS. 10a,10b).

Sequentially, the response of ILC2 to NMU, IL-33 and IL-25, in a dose dependent manner, was compared. Strikingly, when compared to their cytokine counterparts, IL-33 and IL-25, NMU activation of ILC2 led to a prompt, and very robust expression of innate IL-5 and IL-13. This immediate up-regulation of NMU-induced innate type 2 cytokines was comparable to the effects observed with PMA-ionomycin activation, indicating that Neuromedin U is a uniquely potent regulator of ILC2-derived type 2 cytokines (FIGS. 10c,10d).

### Example 8: Effect of NmU23-Induced Cell Activation on NFAT

Neuromedin U-induced activation of ILC2s led to immediate and efficient ERK1/2 activation, while inhibition of ERK activity upon NmU23-induced ILC2 activation resulted in impaired type 2 cytokine gene expression (FIGS. 3a,3b). Analysis of Neuromedin U-induced activation of ILC2s also led to immediate and robust Ca²⁺ influx, suggesting a role of the calcium dependent serine/threonine protein phosphatase Calcineurin in NmU23-induced type 2 responses (FIG. 3c).

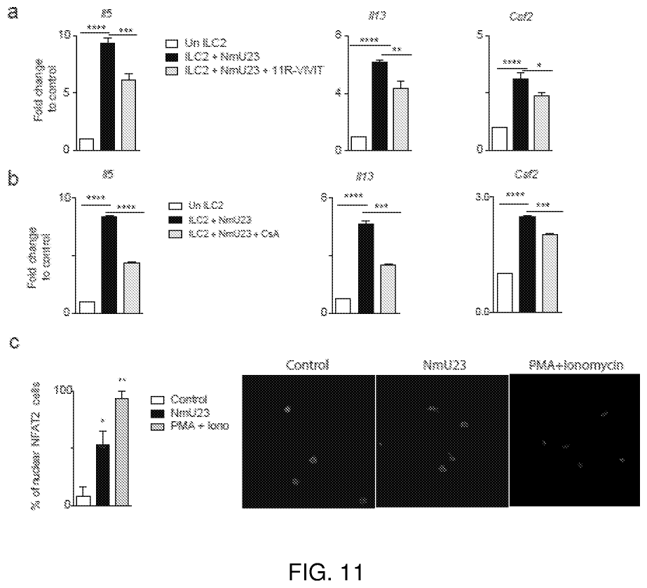

In agreement, inhibition of Calcineurin or its interactions with NFAT, upon NmU23 activation led to impaired innate Il5, Il13 and Csf2 expression (FIG. 3d and FIGS. 11a,11b). In agreement, NmU23-induced cell activation led to efficient translocation of NFAT from the cytoplasm to the nucleus of ILC2 (FIG. 11c). Finally, inhibition of NFAT activity upon NmU23-induced NMUR1 activation led to similarly decreased Il5, Il13 and Csf2 (FIG. 3e). Thus, it was concluded that the neuronal-derived peptide Neuromedin U, can operate in an ILC2-intrinsic manner by activating NMUR1, which regulates innate type 2 cytokine.

### Example 9: Effects of NmU23 Treatment in N. brasiliensis Infected Mice

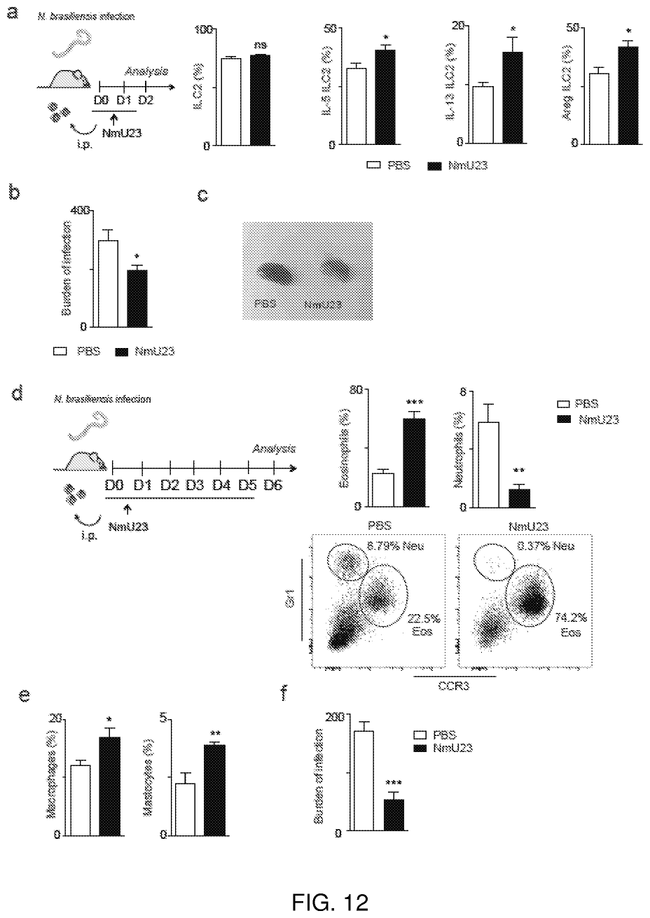

To interrogate whether neuronal peptides regulate mucosal defence, how varying degrees of NMUR1 signals can control mucosal aggression shortly after infection with the helminthic parasite Nippostrongylus brasiliensis, and before adaptive T cell responses are established, was tested. Strikingly, infection of WT mice with N. brasiliensis resulted in strongly increased Nmu expression in the lung (FIG. 4a), suggesting that Neuromedin U may regulate in vivo responses to worm infection. Accordingly, administration of the neuropeptide NmU23 in N. brasiliensis infected mice resulted in a very robust and immediate innate type 2 responses characterised by increased ILC2-derived IL-5, IL-13 and Amphiregulin, and increased eosinophil in the lung when compared to their vehicle (PBS) treated counterparts (FIGS. 4b-4d and FIG. 12a). Accordingly, NmU23 treatment in N. brasiliensis infected mice led to reduced lung haemorrhage and decreased lung and intestinal parasite burden (FIGS. 12b,12f).

### Example 10: Confirmation of the Role of NMUR1 Using Nmur1 Deficient Mice

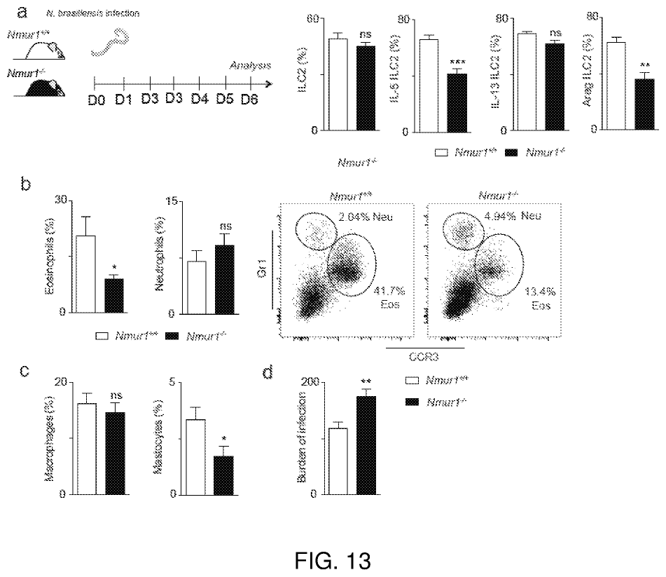

To further explore the role of NMUR1 in innate type 2 responses, Nmur1 deficient mice and their littermate controls were infected with N. brasiliensis (FIGS. 4e-4i). Strikingly, when compared to their WT littermate counterparts, Nmur1 knockout mice had decreased type 2 responses, notably markedly reduced ILC2-derived IL-5, IL-13 and Amphiregulin, reduced eosinophil and granulocyte infiltrates (FIGS. 4e-4g and FIGS. 13a-13c). In line with these findings, Nmur1 deficient mice had increased N. brasiliensis infection burden in the lung and intestine (FIG. 4i and FIG. 13d). Altogether, these data indicate that the neuropeptide Neuromedin U provides critical cues that regulate type 2 responses in vivo, thus increasing immediate mucosal protection against worm infections.

### Example 11: Activation of ILC2 Leads to Innate Type 2 Cytokine Production In Vivo

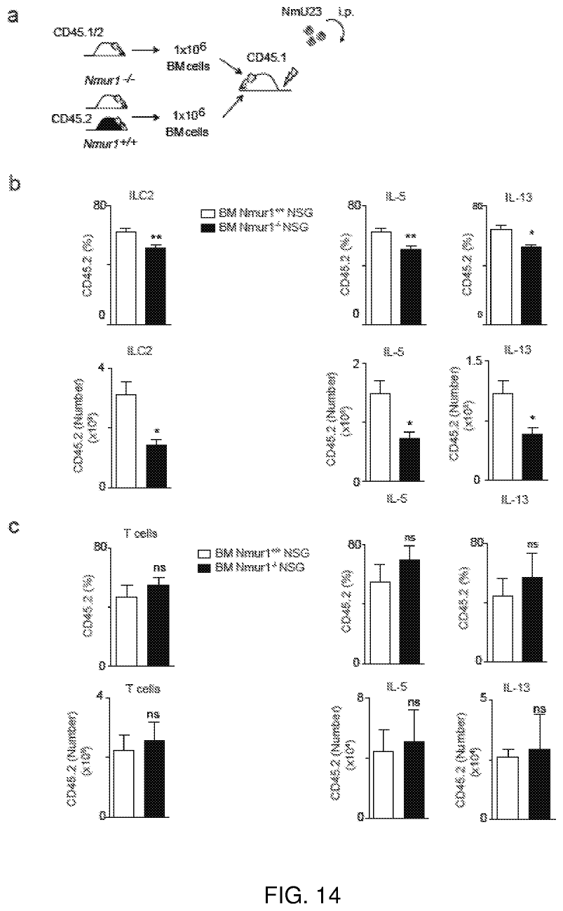

To formally establish the link between ILC2-autonomous activation via NMUR1 and innate type 2 cytokine production in vivo, bone-marrow (BM) mixed chimeras with NMUR1 sufficient and deficient BM cells were performed. It was found that after NMUT administration, Nmur1 deficient ILC2 had reduced innate IL-5 and IL-13 expression when compared to their wild-type competitive counterparts (FIGS. 14a,14b). Noteworthy, Nmur1 deficient or competent T cells had unperturbed expression of these type 2 cytokines (FIG. 14c). Thus, NMU-NMUR1 operate in an ILC2-intrinsic manner to control type 2 cytokine expression in vivo.

### REFERENCES

1 Cording, S., Medvedovic, J., Aychek, T. & Eberl, G. Innate lymphoid cells in defense, immunopathology and immunotherapy. Nat Immunol 17, 755-757 (2016).
2 Klose, C. S. & Artis, D. Innate lymphoid cells as regulators of immunity, inflammation and tissue homeostasis. Nat Immunol 17, 765-774 (2016).
3 Veiga-Fernandes, H. & Mucida, D. Neuro-Immune Interactions at Barrier Surfaces. Cell 165, 801-811 (2016).
4 Ibiza, S. et al. Glial-cell-derived neuroregulators control type 3 innate lymphoid cells and gut defence. Nature 535, 440-443 (2016).
5 van de Pavert, S. A. et al. Maternal retinoids control type 3 innate lymphoid cells and set the offspring immunity. Nature 508, 123-127 (2014).
6 Patel, A. et al. Differential RET signaling pathways drive development of the enteric lymphoid and nervous systems. Sci Signal 5, ra55 (2012).
7 Veiga-Fernandes, H. et al. Tyrosine kinase receptor RET is a key regulator of Peyer's Patch organogenesis. Nature 446, 547-551 (2007).
8 Fonseca-Pereira, D. et al. The neurotrophic factor receptor RET drives haematopoietic stem cell survival and function. Nature 514, 98-101 (2014).
9 Veldhoen, M. & Veiga-Fernandes, H. Feeding immunity: skepticism, delicacies and delights. Nat Immunol 16, 215-219 (2015).
10 Robinette, M. L. et al. Transcriptional programs define molecular characteristics of innate lymphoid cell classes and subsets. Nat Immunol 16, 306-317 (2015).
11 Augood, S. J., Keast, J. R. & Emson, P. C. Distribution and characterisation of neuromedin U-like immunoreactivity in rat brain and intestine and in guinea pig intestine. Regul Pept 20, 281-292 (1988).
12 Ballesta, J. et al. Occurrence and developmental pattern of neuromedin U-immunoreactive nerves in the gastrointestinal tract and brain of the rat. Neuroscience 25, 797-816 (1988).
13 Honzawa, M., Sudoh, T., Minamino, N., Kangawa, K. & Matsuo, H. Neuromedin U-like immunoreactivity in rat intestine: regional distribution and immunohistochemical study. Neuropeptides 15, 1-9 (1990).
14 Martinez, V. G. & O'Driscoll, L. Neuromedin U: a multifunctional neuropeptide with pleiotropic roles. Clin Chem 61, 471-482 (2015).
15 Mulligan, L. M. RET revisited: expanding the oncogenic portfolio. Nat Rev Cancer 14, 173-186 (2014).
16 Hoshi, M., Batourina, E., Mendelsohn, C. & Jain, S. Novel mechanisms of early upper and lower urinary tract patterning regulated by RetY1015 docking tyrosine in mice. Development 139, 2405-2415 (2012).
17 Hermann-Kleiter, N. & Baier, G. NFAT pulls the strings during CD4+ T helper cell effector functions. Blood 115, 2989-2997 (2010).
18 Howard, A. D. et al. Identification of receptors for neuromedin U and its role in feeding. Nature 406, 70-74 (2000).
19 Raddatz, R. et al. Identification and characterization of two neuromedin U receptors differentially expressed in peripheral tissues and the central nervous system. J Biol Chem 275, 32452-32459 (2000).
20 Shan, L. et al. Identification of a novel neuromedin U receptor subtype expressed in the central nervous system. J Biol Chem 275, 39482-39486 (2000).
21 Fallon, P. G. et al. Identification of an interleukin (IL)-25-dependent cell population that provides IL-4, IL-5, and IL-13 at the onset of helminth expulsion. The Journal of experimental medicine 203, 1105-1116 (2006).
22 Van Dyken, S. J. et al. A tissue checkpoint regulates type 2 immunity. Nat Immunol 17, 1381-1387 (2016).
23 Spencer, S. P. et al. Adaptation of innate lymphoid cells to a micronutrient deficiency promotes type 2 barrier immunity. Science 343, 432-437 (2014).
24 Monticelli, L. A. et al. Arginase 1 is an innate lymphoid-cell-intrinsic metabolic checkpoint controlling type 2 inflammation. Nat Immunol 17, 656-665 (2016).
25 Monticelli, L. A. et al. Innate lymphoid cells promote lung-tissue homeostasis after infection with influenza virus. Nat Immunol 12, 1045-1054 (2011).
26 Brestoff, J. R. et al. Group 2 innate lymphoid cells promote beiging of white adipose tissue and limit obesity. Nature 519, 242-246 (2015).
27 Nussbaum, J. C. et al. Type 2 innate lymphoid cells control eosinophil homeostasis. Nature 502, 245-248 (2013).
28 Gabanyi, I. et al. Neuro-immune Interactions Drive Tissue Programming in Intestinal Macrophages. Cell 164, 378-391 (2016).
29 Muller, P. A. et al. Crosstalk between muscularis macrophages and enteric neurons regulates gastrointestinal motility. Cell 158, 300-313 (2014).
30 Irizarry, R. A. et al. Exploration, normalization, and summaries of high density oligonucleotide array probe level data. Biostatistics 4, 249-264 (2003).
31 Gautier, L., Cope, L., Bolstad, B. M. & Irizarry, R. A. affy—analysis of Affymetrix GeneChip data at the probe level. Bioinformatics 20, 307-315 (2004).
32 Huber, W. et al. Orchestrating high-throughput genomic analysis with Bioconductor. Nature methods 12, 115-121 (2015).
33 Ritchie, M. E. et al. limma powers differential expression analyses for RNA-sequencing and microarray studies. Nucleic Acids Res 43, e47 (2015).
34 Bouchery, T. et al. ILC2s and T cells cooperate to ensure maintenance of M2 macrophages for lung immunity against hookworms. Nat Commun 6, 6970 (2015).
35 Joseph, N. M. et al. Enteric glia are multipotent in culture but primarily form glia in the adult rodent gut. The Journal of clinical investigation 121, 3398-3411 (2011).
36 Doherty, T. A. et al. Lung type 2 innate lymphoid cells express cysteinyl leukotriene receptor 1, which regulates TH2 cytokine production. J Allergy Clin Immunol 132, 205-213 (2013).
37 Zhu, J. D. Myeloid cell-lineage and premylocytic-stage-specific-expression of themouse myeloperoxidase gene is controlled at initiation as well as elongation levels of transcription. Cell Res 9, 107-134 (1999).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for increasing activity or proliferation of Group 2 innate lymphoid cells (ILC2s), comprising
contacting ILC2s with an agonist of neuromedin U receptor 1 (NMUR1) in an amount effective to increase activity of the ILC2s.

2. The method of claim 1, wherein the agonist of NMUR1 is neuromedin U (NMU) or an analog thereof, or an antibody that specifically binds and activates NMUR1 or an antigen-binding fragment thereof.

3. The method of claim 2, wherein the NMU or analog thereof is NMU25, NMU precursor protein, NMU23, or NMU8.

4. The method of any one of claims 1-3, wherein the contacting is in vitro.

5. The method of claim 4, wherein the ILC2s are contacted in an ILC2 expansion protocol.

6. The method of any one of claims 1-3, wherein the contacting is in vivo.

7. The method of claim 6, wherein the agonist of neuromedin U receptor 1 (NMUR1) is administered to a subject.

8. The method of claim 7, wherein the subject is a human.

9. The method of claim 7 or claim 8, wherein the subject is not otherwise in need of treatment with the agonist of NMUR1.

10. A method for treating a disease associated with Group 2 innate lymphoid cells (ILC2s), comprising
administering to a subject in need of such treatment an agonist of neuromedin U receptor 1 (NMUR1) in an amount effective to treat the disease.

11. The method of claim 10, wherein the agonist of NMUR1 is neuromedin U (NMU) or an analog thereof, or an antibody that specifically binds and activates NMUR1 or an antigen-binding fragment thereof.

12. The method of claim 11, wherein the NMU or analog thereof is NMU25, NMU precursor protein, NMU23, or NMU8.

13. The method of any one of claims 10-12, wherein the subject is a human.

14. The method of any one of claims 10-13, wherein the disease is infection, tissue repair, wound healing, obesity, treatable by increasing induction of type 2 immune responses, treatable by metabolic regulation, treatable by increasing eosinophils, or treatable by increasing mast cells.

15. The method of any one of claims 10-14, wherein the subject is not otherwise in need of treatment with the agonist of NMUR1.

16. The method of any one of claims 10-15, wherein the agonist of NMUR1 is administered intravenously, orally, nasally, rectally or through skin absorption.

17. An agonist of neuromedin U receptor 1 (NMUR1) for use in treating a disease associated with Group 2 innate lymphoid cells (ILC2s), comprising administering to a subject in need of such treatment the agonist of NMUR1 in an amount effective to treat the disease.

18. The agonist of claim 17, wherein the agonist of NMUR1 is neuromedin U (NMU) or an analog thereof, or an antibody that specifically binds and activates NMUR1 or an antigen-binding fragment thereof.

19. The agonist of claim 18, wherein the NMU or analog thereof is NMU25, NMU precursor protein, NMU23, or NMU8.

20. The agonist of any one of claims 17-19, wherein the subject is a human.

21. The agonist of any one of claims 17-20, wherein the disease is infection, tissue repair, wound healing, obesity, treatable by increasing induction of type 2 immune responses, treatable by metabolic regulation, treatable by increasing eosinophils, or treatable by increasing mast cells.

22. The agonist of any one of claims 17-21, wherein the subject is not otherwise in need of treatment with the agonist of NMUR1.

23. The agonist of any one of claims 17-22, wherein the agonist of NMUR1 is administered intravenously, orally, nasally, rectally or through skin absorption.

24. A method for treating a disease associated with Group 2 innate lymphoid cells (ILC2s), comprising
administering to a subject in need of such treatment a composition comprising activated ILC2s in an amount effective to treat the disease.

25. The method of claim 24, wherein the composition further comprises an agonist of neuromedin U receptor 1 (NMUR1).

26. The method of claim 25, wherein the agonist of NMUR1 is neuromedin U (NMU) or an analog thereof, or an antibody that specifically binds and activates NMUR1 or an antigen-binding fragment thereof.

27. The method of claim 26, wherein the NMU or analog thereof is NMU25, NMU precursor protein, NMU23, or NMU8.

28. The method of any one of claims 24-27, wherein the subject is a human.

29. The method of any one of claims 24-28, wherein the disease is infection, tissue repair, wound healing, obesity, treatable by increasing induction of type 2 immune responses, treatable by metabolic regulation, treatable by increasing eosinophils, or treatable by increasing mast cells.

30. The method of any one of claims 24-29, wherein the subject is not otherwise in need of treatment with the activated ILC2s or the agonist of NMUR1.

31. The method of any one of claims 24-30, wherein the activated ILC2s or the agonist of NMUR1 is administered intravenously, orally, nasally, rectally or through skin absorption.

32. A composition comprising activated Group 2 innate lymphoid cells (ILC2s) for use in treating a disease associated with ILC2s comprising administering to a subject in need of such treatment the composition comprising activated ILC2s in an amount effective to treat the disease.

33. The composition of claim 32, wherein the composition further comprises an agonist of neuromedin U receptor 1 (NMUR1).

34. The composition of claim 33, wherein the agonist of NMUR1 is neuromedin U (NMU) or an analog thereof, or an antibody that specifically binds and activates NMUR1 or an antigen-binding fragment thereof.

35. The composition of claim 34, wherein the NMU or analog thereof is NMU25, NMU precursor protein, NMU23, or NMU8.

36. The composition of any one of claims 32-35, wherein the subject is a human.

37. The composition of any one of claims 32-36, wherein the disease is infection, tissue repair, wound healing, obesity, treatable by increasing induction of type 2 immune responses, treatable by metabolic regulation, treatable by increasing eosinophils, or treatable by increasing mast cells.

38. The composition of any one of claims 32-37, wherein the subject is not otherwise in need of treatment with the activated ILC2s or the agonist of NMUR1.

39. The composition of any one of claims 32-38, wherein the activated ILC2s or the activated ILC2s and the agonist of NMUR1 is administered intravenously, orally, nasally, rectally or through skin absorption.

40. A method for decreasing activity or proliferation of Group 2 innate lymphoid cells (ILC2s), comprising
contacting ILC2s with an antagonist of neuromedin U receptor 1 (NMUR1) or neuromedin U (NMU) in an amount effective to decrease activity of the ILC2s.

41. The method of claim 40, wherein the antagonist of NMUR1 or NMU is an antibody that specifically binds and inhibits NMUR1 or NMU, respectively, or an antigen-binding fragment thereof.

42. The method of claim 40, wherein the antagonist of NMUR1 or NMU is an inhibitory nucleic acid molecule that reduces that reduces expression, transcription or translation of NMUR1 or NMU.

43. The method of claim 42, wherein the inhibitory nucleic acid is a sRNA, shRNA, or antisense nucleic acid molecule.

44. The method of any one of claims 40-43, wherein the contacting is in vitro.

45. The method of any one of claims 40-43, wherein the contacting is in vivo.

46. The method of claim 45, wherein the antagonist of NMUR1 or NMU is administered to a subject.

47. The method of claim 46, wherein the subject is a human.

48. The method of claim 46 or claim 47, wherein the subject is not otherwise in need of treatment with the antagonist of NMUR or NMU 1.

49. A method for treating a disease associated with Group 2 innate lymphoid cells (ILC2s), comprising
administering to a subject in need of such treatment an antagonist of neuromedin U receptor 1 (NMUR1) or neuromedin U (NMU) in an amount effective to treat the disease.

50. The method of claim 49, wherein the antagonist of NMUR1 or NMU is an antibody that specifically binds and inhibits NMUR1 or NMU, respectively, or an antigen-binding fragment thereof.

51. The method of claim 49, wherein the antagonist of NMUR1 or NMU is an inhibitory nucleic acid molecule that reduces that reduces expression, transcription or translation of NMUR1 or NMU.

52. The method of claim 51, wherein the inhibitory nucleic acid is a sRNA, shRNA, or antisense nucleic acid molecule.

53. The method of any one of claims 49-52, wherein the subject is a human.

54. The method of any one of claims 49-53, wherein the disease is allergy, allergic asthma, food allergy, eosinophilic esophagitis, atopic dermatitis, fibrosis, allergic rhinitis, allergic rhinosinusitis, chronic obstructive pulmonary disease (COPD), cystic fibrosis, treatable by reducing type 2 immune responses, treatable by reducing eosinophils, or treatable by reducing mast cells.

55. The method of any one of claims 49-54, wherein the subject is not otherwise in need of treatment with the agonist of NMUR1 or NMU.

56. The method of any one of claims 49-55, wherein the antagonist of NMUR1 is administered intravenously, orally, nasally, rectally or through skin absorption.

57. An antagonist of neuromedin U receptor 1 (NMUR1) or neuromedin U (NMU) for use in treating a disease associated with Group 2 innate lymphoid cells (ILC2s) comprising administering to a subject in need of such treatment the antagonist of NMUR1 or NMU in an amount effective to treat the disease.

58. The antagonist of claim 57, wherein the antagonist of NMUR1 or NMU is an antibody that specifically binds and inhibits NMUR1 or NMU, respectively, or an antigen-binding fragment thereof.

59. The antagonist of claim 57, wherein the antagonist of NMUR1 or NMU is an inhibitory nucleic acid molecule that reduces that reduces expression, transcription or translation of NMUR1 or NMU.

60. The antagonist of claim 59, wherein the inhibitory nucleic acid is a sRNA, shRNA, or antisense nucleic acid molecule.

61. The antagonist of any one of claims 57-60, wherein the subject is a human.

62. The antagonist of any one of claims 57-61, wherein the disease is allergy, allergic asthma, food allergy, eosinophilic esophagitis, atopic dermatitis, fibrosis, allergic rhinitis, allergic rhinosinusitis, chronic obstructive pulmonary disease (COPD), cystic fibrosis, treatable by reducing type 2 immune responses, treatable by reducing eosinophils, or treatable by reducing mast cells.

63. The antagonist of any one of claims 57-62, wherein the subject is not otherwise in need of treatment with the agonist of NMUR1 or NMU.

64. The antagonist of any one of claims 57-63, wherein the antagonist of NMUR1 or NMU is administered intravenously, orally, nasally, rectally or through skin absorption.


SEQUENCE LISTING (6 sequences)

SEQ ID NO: 1 - protein, 174 aa, Homo sapiens
MLRTESCRPRSPAGQVAAASPLLLLLLLLAWCAGACRGAPILPQGLQPEQQLQLWNEIDDTCSSFLSIDSQPQASNALEELCFMIMGMLPKPQEQDEKDNTKRFLFHYSKTQKLGKSNVVSSVVHPLLQLVPHLHERRMKRFRVDEEFQSPFASQSRGYFLFRPRNGRRSAGFI

SEQ ID NO: 2 - protein, 25 aa, Homo sapiens
FRVDEEFQSPFASQSRGYFLFRPRN

SEQ ID NO: 3 - protein, 23 aa, Mus musculus
FKAEYQSPSVGQSKGYFLFRPRN

SEQ ID NO: 4 - protein, 8 aa, Homo sapiens
YFLFRPRN

SEQ ID NO: 5 - protein, 33 aa, Artificial Sequence
  Analog of Neuromedin U
ILQRGSGTAAVDFTKKDHTATWGRPFFLFRPRN

SEQ ID NO: 6 - protein, 5 aa, Artificial Sequence
  Neuromedin U inhibitory peptide
FRPRN